(19) United States
(12) Patent Application Publication
(10) Pub. No.: US 2009/0163481 A1
(43) Pub. Date: Jun. 25, 2009

(54) PPAR-DELTA LIGANDS AND METHODS OF THEIR USE

(76) Inventors: Brian J. Murphy, Redwood City, CA (US); Nurulain T. Zaveri, Saratoga, CA (US); Barbara G. Sato, Castro Valley, CA (US); Faming Jiang, Mountain View, CA (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/335,377

(22) Filed: Dec. 15, 2008

Related U.S. Application Data
(60) Provisional application No. 61/007,786, filed on Dec. 13, 2007.

(51) Int. Cl. A61K 31/5415; A61K 31/426; A61K 31/192; A61K 31/403; A61P 29/00; A61P 35/00; A61P 9/00; A61P 3/00; A61P 17/02

(52) U.S. Cl. 514/226.2; 514/365; 514/569; 514/411; 514/568

(57) ABSTRACT

The disclosure provides compounds, compositions, and methods for modulating PPARδ receptor. In one embodiment, the compounds of the disclosure comprise a tri-substituted thiazole group. The substituent at the 2-position of the thiazole group provides steric bulk to the compounds. The compounds, compositions, and methods may be useful, for example, in the treatment of cancer.

### CROSS-REFERENCE TO RELATED APPLICATION

This application claims priority under 35 U.S.C. § 119(e)(1) to U.S. Provisional Patent Application Ser. No. 61/007,786, filed Dec. 13, 2007, the contents of which are incorporated herein by reference.

### GOVERNMENT RIGHTS

This invention was made in part with government support under grant number DAMD17-02-1-0141 awarded by the U.S. Army. The government has certain rights in this invention.

### TECHNICAL FIELD

This invention relates generally to compounds and compositions effective for modulating PPAR-delta, as well as methods of treating conditions associated with PPARδ. The invention finds utility, for example, in the field of medicine.

### BACKGROUND

Peroxisome proliferator activated receptors (PPARs) are ligand-activated transcription factors belonging to the nuclear hormone receptor family that also includes the androgen receptor (AR). Certain fatty acids and fatty acid metabolites are believed to be endogenous ligands of these transcription factors. Three isotypes (PPARα, γ, and δ), displaying distinct tissue distribution and functions, have been identified. Studies show that PPARδ is a multifunctional transcription factor controlling not only fat catabolism, but also many diverse physiological and pathological processes, including embryonic development, inflammation, wound healing, cardiovascular diseases, and tumor development. PPARδ is probably involved in the development of colorectal carcinomas.

Several endogenous ligands, including polyunsaturated fatty acids (PUFAs) and eicosanoids, have been shown to activate PPARδ with micromolar affinity. Among the eicosanoids, PGA1 was the first to be described as an activator of PPARδ. The naturally occurring prostacyclin (PGI2), a product of cyclooxygenase-2 (COX-2)-mediated eicosanoid synthesis from arachidonic acid, and its semisynthetic analog carbaprostacyclin (cPGI) have been reported as being among the more selective activators of PPARδ. In addition, PGE2 (known to be elevated in a number of different tumor types) is also a potent activator of PPARδ, but through an indirect pathway involving the PI3-kinase/Akt pathway. Among synthetic ligands, a high-affinity but nonselective agonist, GW 2433, is a dual activator of PPARδ and PPARδ. This ligand is nonselective.

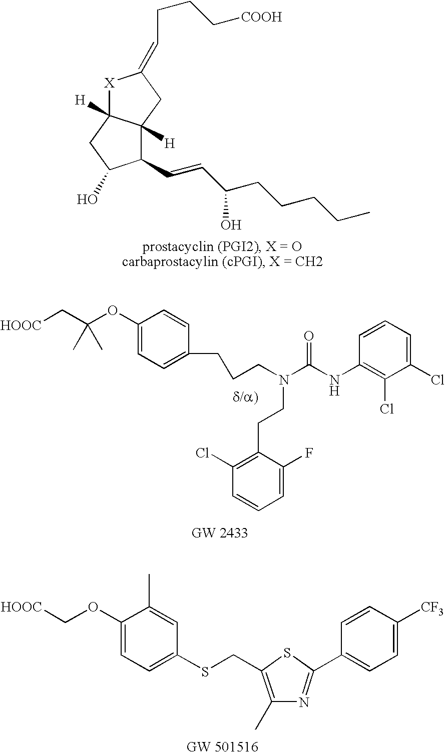

A selective and potent synthetic PPARδ agonist, GW 501516 (GW-501), has in excess of 100-fold selectivity for PPARδ over the PPARγ and a receptors. This ligand is an activator of PPARδ.

Some information is available concerning PPARδ biology, the importance of PPARδ in both metabolic syndrome and tumorigenesis, and the use of small molecule activators in normal and diseased tissues. Nevertheless, a thorough analysis of the importance of PPARδ in a variety of biological processes is not available in the relevant literature, and ligands for PPARδ remain desirable targets in synthetic and medicinal chemistry. Ideally, ligands would be simple to prepare and tunable in the sense that the PPARδ-modulating properties of the ligands would be controlled via structural modifications.

The present invention is directed at addressing one or more of the abovementioned drawbacks and desired features, as well as related issues in the field of medicinal chemistry.

### SUMMARY OF THE INVENTION

In some embodiments, then, the invention provides a method for modulating a PPAR-δ receptor. The method comprises administering a compound having the structure of formula (I):

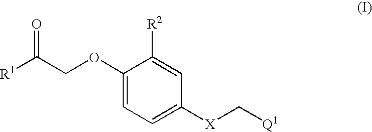

wherein: R¹ is selected from —OR³ and N(R⁴)(R⁵); R² is hydrocarbyl; R³ is selected from H and hydrocarbyl; R⁴ and R⁵ are independently selected from H and hydrocarbyl; X is selected from —S—, —O—, and —NR⁸—, where R⁸ is selected from H and hydrocarbyl; Q¹ is —(CH₂)ₙ-Q²-B; n is an integer from 0 to 3; Q² is selected from a bond, —O—, —C(═O)—NR⁷—, and

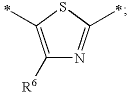

R⁶ is hydrocarbyl; R⁷ is selected from H, alkyl, aryl, alkaryl, and aralkyl, any of which may be unsubstituted or substituted; and B is a bulk-providing group.

In other embodiments, the invention provides compounds having the structure of formula (I).

In other embodiments, the invention provides a method for modulating a PPAR-δ receptor. The method comprises administering a compound comprising a trisubstituted thiazole group, wherein the substituent at the 2-position of the thiazole group is a bulk-providing group that is sterically larger than a 4-(trifluoromethyl)phenyl group.

These and other aspects of the invention are described herein.

### BRIEF DESCRIPTION OF THE DRAWINGS

FIG. 1 provides graphical data showing Ligand binding domain (1A-1 through 1A-6) and PPRE transactivation (1B-1 through 1B-5) analyses of selected ligands.

FIG. 2 shows Western analysis of expression of PPARδ and GAPDH (control) in various cancer cells.

FIG. 3 provides graphical data showing PPARδ ligand binding (A) and transactivation analysis (B) of VLDL (±SR13904).

FIG. 4 shows Western analysis of cyclin D1 and CDK2 as functions of PPAR-δ modulation.

FIG. 5A provides graphical data showing that a compound according to the invention exerts inhibitory effects on the cell cycle

FIG. 5B provides a statistical analysis of cell cycle distribution.

FIG. 5C provides data showing select cell cycle protein levels over time.

FIG. 5D provides mRNA analysis of CDK2, CKD4, and cyclic D1.

FIG. 6 provides data showing that PPARδ modulates drug-induced apoptosis.

FIG. 7 shows Gelatin zymogel analysis of MMP-9 as a function of PPARδ activation.

FIG. 8 provides data showing that PPARδ regulates the Akt1 pathway in cancer cells.

### DETAILED DESCRIPTION OF THE INVENTION

### Definitions and Nomenclature

Before describing the present invention in detail, it is to be understood that unless otherwise indicated, this invention is not limited to particular compounds, compositional forms, synthetic methods, or methods of use, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, “a PPARδ antagonist” refers not only to a single PPARδ antagonist but also to a combination of two or more different antagonists, “an excipient” refers to a combination of excipients as well as to a single excipient, and the like.

As used herein, the phrases “for example,” “for instance,” “such as,” and “including” are meant to introduce examples that further clarify more general subject matter. These examples are provided only as an aid for understanding the disclosure, and are not meant to be limiting in any fashion.

Unless defined otherwise, all technical and scientific terms used herein have the meaning commonly understood by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Reference to specific alkyl groups is meant to include all constitutional isomers that exist for that group. Generally, although again not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and the terms “heteroatom-containing alkyl” and “heteroalkyl” refer to an alkyl substituent in which at least one carbon atom is replaced with a heteroatom, as described in further detail infra. Substituted alkyl includes, for example, instances where two hydrogen atoms on an alkyl carbon atom have been replaced with a pi-bonded oxygen, such that a carbonyl (C═O) group is formed. If not otherwise indicated, the terms “alkyl” and “lower alkyl” include linear, branched, cyclic, unsubstituted, substituted, and/or heteroatom-containing alkyl or lower alkyl, respectively.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein may, for example, may contain 1 to 3 carbon atoms, and as a further example, such substituents may contain 1 or 2 carbon atoms (i.e., methoxy and ethoxy).

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “amino” is used herein to refer to the group —NZ¹Z² wherein Z¹ and Z² are hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

The term “cyclic” as used herein refers to a molecule, linkage, or substituent, that is or includes a circular connection or atoms. Unless otherwise indicated, the term “cyclic” includes aromatic, alicyclic, substituted, unsubstituted, heteroatom-containing moieties, and combinations thereof.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—C(═O)-alkyl) and C₆-C₂₀ arylcarbonyl (—C(═O)-aryl)), acyloxy (—O-acyl), C₂-C₂₄ alkoxycarbonyl (—C(═O)—O-alkyl), C₆-C₂₀ aryloxycarbonyl (—C(═O)—O-aryl), halocarbonyl (—C(═O)—X where X is halo), C₂-C₂₄ alkylcarbonato (—O—C(═O)—O-alkyl), C₆-C₂₀ arylcarbonato (—O—C(═O)—O-aryl), carboxy (—COOH), carboxylato (—COO—), carbamoyl (—C(═O)—NH₂), mono-substituted C₁-C₂₄ alkylcarbamoyl (—C(═O)—NH(C₁-C₂₄ alkyl)), di-substituted alkylcarbamoyl (—C(═O)—N(C₁-C₂₄ alkyl)₂), mono-substituted arylcarbamoyl (—C(═O)—NH-aryl), thiocarbamoyl (—C(═S)—NH₂), carbamido (—NH—C(═O)—NH₂), cyano (—C≡N), isocyano (—N⁺—C⁻), cyanato (—O—C≡N), isocyanato (—O—N⁺═C⁻), isothiocyanato (—S—C≡N), azido (—N═N⁺═N⁻), formyl (—C(═O)—H), thioformyl (—C(═S)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—C(═O)-alkyl), C₅-C₂₀ arylamido (—NH—C(═O)-aryl), imino (—CR═NH where R=hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—S(O)-alkyl), C₅-C₂₀ arylsulfinyl (—S(O)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C₁-C₂₄ alkyl)-substituted phosphino, mono- and di-(C₅-C₂₀ aryl)-substituted phosphino; and the hydrocarbyl moieties C₁-C₂₄ alkyl (including C₁-C₁₈ alkyl, further including C₁-C₁₂ alkyl, and further including C₁-C₆ alkyl), C₂-C₂₄ alkenyl (including C₂-C₁₈ alkenyl, further including C₂-C₁₂ alkenyl, and further including C₂-C₆ alkenyl), C₂-C₂₄ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₃₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated. Where appropriate and unless otherwise specified, the terms “substituted” and “substituent” when used in the context of cyclic groups such as aromatic and alicyclic groups are meant to include fused rings and other multiple ring systems. For example, a substituted aryl group includes such groups as naphthyl and anthracenyl.

When the term “substituted” appears prior to a list of possible substituted groups, it is intended that the term apply to every member of that group. For example, the phrase “substituted alkyl and aryl” is to be interpreted as “substituted alkyl and substituted aryl.”

By two moieties being “connected” is intended to include instances wherein the two moieties are directly bonded to each other, as well as instances wherein a linker moiety (such as an alkylene or heteroatom) is present between the two moieties.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Unless otherwise indicated, the terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. “Preventing” a disorder or unwanted physiological event in a patient refers specifically to the prevention of the occurrence of symptoms and/or their underlying cause, wherein the patient may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the individual, the particular beneficial agent or agents, and the like. Thus, it is not always possible to specify an exact “effective amount.” However, an appropriate “effective” amount in any individual case may be determined by one of ordinary skill in the art using routine experimentation.

As used herein, and unless specifically stated otherwise, an “effective amount” of a beneficial refers to an amount covering both therapeutically effective amounts and prophylactically effective amounts.

As used herein, a “therapeutically effective amount” of an active agent refers to an amount that is effective to achieve a desired therapeutic result, and a “prophylactically effective amount” of an active agent refers to an amount that is effective to prevent or lessen the severity of an unwanted physiological condition.

By a “pharmaceutically acceptable” component is meant a component that is not biologically or otherwise undesirable, i.e., the component may be incorporated into a pharmaceutical formulation and administered to a patient as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “pharmacologically active” (or simply “active”), as in a “pharmacologically active” derivative or analog, refers to a derivative or analog (e.g., a salt, ester, amide, conjugate, metabolite, isomer, fragment, etc.) having the same type of pharmacological activity as the parent compound and approximately equivalent in degree.

The term “controlled release” refers to a formulation, dosage form, or region thereof from which release of a beneficial agent is not immediate, i.e., with a “controlled release” dosage form, administration does not result in immediate release of the beneficial agent in an absorption pool. The term is used interchangeably with “non-immediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a formulation, dosage form, or region thereof that provides for gradual release of a beneficial agent over an extended period of time, and that preferably, although not necessarily, results in substantially constant blood levels of the agent over an extended time period.

The term “naturally occurring” refers to a compound or composition that occurs in nature, regardless of whether the compound or composition has been isolated from a natural source or chemically synthesized.

A compound may exhibit “selective” binding, by which is meant that the compound's affinity for binding to one or more particular receptor(s) is greater than the compound's affinity for binding to one other receptor, multiple other receptor, or all other receptors. For a compound that exhibits selective binding, therefore, the binding constant Kᵢ for the compound binding with one receptor is lower than the Kᵢ for the compound binding with one or more other receptor(s). For example, a compound that is selective for receptor “A” over receptor “B” will have a binding constant ratio Kᵢ(A)/Kᵢ(B) that is less than 1/1.

The terms “van der Waals radius” and “van der Waals volume” are used herein to quantify the physical size of various atoms and collections of atoms. The terms are synonymous with “atomic radius” and “atomic volume,” respectively. Because such terms refer to physical constructs, and because typical values of van der Waals radii and van der Waals volumes may vary, the following values will be exclusively used herein (values in A): hydrogen=1.20, carbon=1.70, nitrogen=1.55, oxygen=1.52, fluorine=1.47, phosphorus=1.80, sulfur=1.80, chlorine=1.75, bromine=1.85, iodine=1.98, and silicon=2.10. Additional atomic radii can be found in Bondi (1964) J. Phys. Chem., 68, 441. Using these radii values and the equation V=(4/3)π*r³, the atomic volumes used herein are as follows (values in Å³): hydrogen=7.2, carbon=20.6, nitrogen=15.6, oxygen=14.7, fluorine=13.3, phosphorus=24.4, sulfur=24.4, chlorine=22.4, bromine=26.5, iodine=32.5, and silicon=38.8. When referring to a chemical compound or a chemical substituent, the term “molecular volume” as used herein refers to the summation of the atomic volumes of the atoms in the compound or substituent. For example, the molecular volume of methane is 49.4 Å³, which is the summation of the atomic volumes of carbon and four hydrogen atoms (i.e., 20.6+4*7.2). Similarly, the molecular volume of a trifluoromethyl substituent is 60.5 Å³, the molecular volume for a trifluoromethoxy substituent is 75.2 Å³, the molecular volume of a 4-(trifluoromethyl)phenyl group is 212.9 Å³, and the molecular volume of a —CH═CH—CH═CH— group (i.e., a fused ring substituent on a cyclic compound) is 111.2 Å³. It will be appreciated that this definition allows substituents and compounds to be conveniently compared on the basis of volume. Thus, a first substituent may be described as “sterically larger” or “sterically smaller” than a second substituent. By “sterically larger” or “sterically smaller” is meant that the first group has a van der Waals volume that is larger or smaller, respectively, than the van der Waals volume of the second group (as calculated using the above procedure).

The compounds described herein are modulators of PPARδ, and are preferably selected from antagonists and agonists of PPARδ. In some embodiments, the compounds described herein are antagonists of PPARδ. Thus, the compounds bind to, but do not modulate, the PPARδ receptor. In other embodiments, the compounds described herein are agonists of PPARδ.

In some embodiments, the compound of the invention have the structure of formula (I):

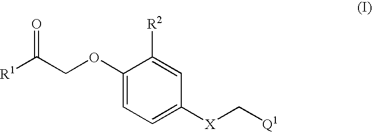

wherein:

R¹ is selected from —OR³ and —N(R⁴)(R⁵);

R² is hydrocarbyl;

R³ is selected from H and hydrocarbyl;

R⁴ and R⁵ are independently selected from H and hydrocarbyl;

X is selected from —S—, —O—, and —NR⁸—;

R⁸ is selected from H and hydrocarbyl;

Q¹ is —(CH₂)ₙ-Q²-B

n is an integer from 0 to 3

Q² is selected from a bond, —O—, —C(═O)—NR⁷—, and

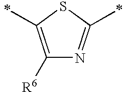

R⁶ is hydrocarbyl;

R⁷ is selected from H, alkyl, aryl, alkaryl, and aralkyl, any of which may be unsubstituted or substituted; and

B is a bulk-providing group.

For example, R² is selected from substituted or unsubstituted C₁-C₂₄ alkyl, substituted or unsubstituted heteroatom containing C₁-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted or unsubstituted C₅-C₂₄ aryl, substituted or unsubstituted C₅-C₂₄ heteroaryl, substituted or unsubstituted C₅-C₂₄ alkaryl, substituted or unsubstituted C₅-C₂₄ aralkyl, substituted or unsubstituted C₅-C₂₄ heteroaralkyl, and substituted or unsubstituted C₅-C₂₄ heteroalkaryl. In some embodiments, R² is C₁-C₁₂ alkyl, for example lower alkyl. In some embodiments, R² is propyl, ethyl, or methyl.

Also for example, R³ is selected from H, substituted or unsubstituted C₁-C₂₄ alkyl, substituted or unsubstituted heteroatom containing C₁-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted or unsubstituted C₅-C₂₄ aryl, substituted or unsubstituted C₅-C₂₄ heteroaryl, substituted or unsubstituted C₅-C₂₄ alkaryl, substituted or unsubstituted C₅-C₂₄ aralkyl, substituted or unsubstituted C₅-C₂₄ heteroaralkyl, and substituted or unsubstituted C₅-C₂₄ heteroalkaryl. In some embodiments, R³ is C₁-C₁₂ alkyl, for example lower alkyl. In some embodiments, R³ is butyl, propyl, ethyl, or methyl.

Also for example, R⁴ and R⁵ are independently selected from H, substituted or unsubstituted C₁-C₂₄ alkyl, substituted or unsubstituted heteroatom containing C₁-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted or unsubstituted C₅-C₂₄ aryl, substituted or unsubstituted C₅-C₂₄ heteroaryl, substituted or unsubstituted C₅-C₂₄ alkaryl, substituted or unsubstituted C₅-C₂₄ aralkyl, substituted or unsubstituted C₅-C₂₄ heteroaralkyl, and substituted or unsubstituted C₅-C₂₄ heteroalkaryl. In some embodiments, R⁴ and R⁵ are C₁-C₁₂ alkyl or C₅-C₁₂ aryl.

Also for example, R⁶ is selected from H, substituted or unsubstituted C₁-C₂₄ alkyl, substituted or unsubstituted heteroatom containing C₁-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted or unsubstituted C₅-C₂₄ aryl, substituted or unsubstituted C₅-C₂₄ heteroaryl, substituted or unsubstituted C₅-C₂₄ alkaryl, substituted or unsubstituted C₅-C₂₄ aralkyl, substituted or unsubstituted C₅-C₂₄ heteroaralkyl, and substituted or unsubstituted C₅-C₂₄ heteroalkaryl. In some embodiments, R⁸ is C₁-C₁₂ alkyl or C₅-C₁₂ aryl.

Also for example, R⁶ is selected from substituted or unsubstituted C₁-C₂₄ alkyl, substituted or unsubstituted heteroatom containing C₁-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted or unsubstituted C₅-C₂₄ aryl, substituted or unsubstituted C₅-C₂₄ heteroaryl, substituted or unsubstituted C₅-C₂₄ alkaryl, substituted or unsubstituted C₅-C₂₄ aralkyl, substituted or unsubstituted C₅-C₂₄ heteroaralkyl, and substituted or unsubstituted C₅-C₂₄ heteroalkaryl. In some embodiments, R⁶ is C₁-C₁₂ alkyl, for example lower alkyl. In some embodiments, R⁶ is butyl, propyl, ethyl, or methyl.

Also for example, R⁷ is selected from H, substituted or unsubstituted C₁-C₂₄ alkyl, substituted or unsubstituted heteroatom containing C₁-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted or unsubstituted C₅-C₂₄ aryl, substituted or unsubstituted C₅-C₂₄ heteroaryl, substituted or unsubstituted C₅-C₂₄ alkaryl, substituted or unsubstituted C₅-C₂₄ aralkyl, substituted or unsubstituted C₅-C₂₄ heteroaralkyl, and substituted or unsubstituted C₅-C₂₄ heteroalkaryl. In some embodiments, R⁷ is selected from H, substituted or unsubstituted C₁-C₁₂ alkyl, substituted or unsubstituted C₅-C₁₂ aryl, substituted or unsubstituted C₅-C₁₂ alkaryl, and substituted or unsubstituted C₅-C₁₂ aralkyl. In some embodiments, R⁷ is H. In some embodiments, R⁷ is unsubstituted aralkyl, or aralkyl substituted with one or more substituents selected from halo, hydroxyl, lower alkyl, and lower alkoxy. In some embodiments, R⁷ is aralkyl substituted with one, two, or three substituents selected from chloro, fluoro, and methoxy.

In some embodiments, the bulk-providing group B is a group that is sterically larger than a 4-(trifluoromethyl)phenyl group, or is sterically larger than a 4-[4-(trifluoromethyl)phenyl]phenyl group. For example, B may have a van der Waals volume greater than 100 Å³, greater than 150 Å³, greater than 200 Å³, greater than 220 Å³, greater than 240 Å³, greater than 250 Å³, greater than 300 Å³, greater than 400 Å³, or greater than 500 Å³, when calculated using the method described hereinabove.

In some embodiments, B is a cyclic group that may be aromatic or alicyclic. In some embodiments, B is a ring system having one or more substituents and/or two or more fused rings. In preferred embodiments, the one or more substituents are other than trifluoromethyl. For example, the one or more substituents may be alkyl or alkoxy substituents.

In some embodiments, B is a fused ring system comprising two or more fused rings, any of which may be aromatic. Examples of such ring systems include those having the structure

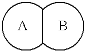

wherein A and B each represent 4-, 5-, 6-, 7-, or 8-membered rings that may be saturated, unsaturated, and/or aromatic. Furthermore, A and B may each contain 0, 1, 2, 3 or more heteroatoms, and 0, 1, 2, 3 or more substituents, and may be further fused to one or more additional rings.

Fused ring systems that are suitable as bulk-providing groups include the following examples of fused 5- and 6-membered ring systems (dotted lines represent optional double bonds):

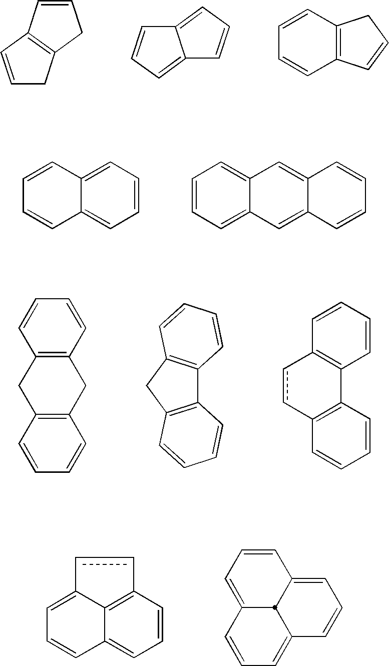

It will be appreciated that the groups shown above and other fused ring systems may be further substituted, may contain one or more heteroatoms, and may be connected to the remained of the compound at any appropriate location. Thus, for example, B may be 1-naphthyl or 2-naphthyl, or B may be 2-quinolinyl, 3-quinolinyl, 4-quinolinyl, 5-quinolinyl, 6-quinolinyl, 7-quinolinyl, or 8-quinolinyl. Examples of substituents include, for example, halo, hydroxyl, alkyl (including halogenated alkyl), alkoxy (including halogenated alkoxy), aryl, and aryloxy.

In some embodiments B is substituted or unsubstituted aryl, B has the structure

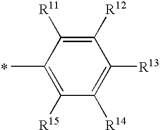

wherein R¹¹-R¹⁵ are H or non-hydrogen substituents. In some embodiments, at least two of R¹¹-R¹⁵ are linked to form a cycle. In preferred embodiments, each of R¹¹-R¹⁵ are independently selected from substituted or unsubstituted C₁-C₂₄ alkyl, substituted or unsubstituted heteroatom containing C₁-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted or unsubstituted C₅-C₂₄ aryl, substituted or unsubstituted C₅-C₂₄ heteroaryl, substituted or unsubstituted C₅-C₂₄ alkaryl, substituted or unsubstituted C₅-C₂₄ aralkyl, substituted or unsubstituted C₅-C₂₄ heteroaralkyl, and substituted or unsubstituted C₅-C₂₄ heteroalkaryl.

In some embodiments, R¹¹-R¹⁵ are selected from substituted or unsubstituted C₂-C₂₄ alkyl (for example substituted or unsubstituted C₇-C₂₄ alkyl), substituted or unsubstituted heteroatom containing C₂-C₂₄ alkyl (for example substituted or unsubstituted heteroatom containing C₄-C₂₄ alkyl), and substituted or unsubstituted heteroatom containing C₅-C₂₄ alkyl.

In some embodiments, R¹¹-R¹⁵ are non-hydrogen substituents other than halogen, —CF₃, —OCH₃, —CH₃, C₁-C₆ straight or branched alkyl, C₁-C₄ alkoxy, —C(O)(C₁-C₄)alkyl, —O—(C₁-C₂)alkyl-CO₂H, aryloxy, arylthio, [1,3,2]dioxaborolanyl, pyridinyl, pyrimidinyl, pyrazinyl, or aryl(C₀-C₄)alkyl. For example, R¹¹-R¹⁵ are substituted or unsubstituted C₇-C₂₄ alkyl, substituted or unsubstituted heteroatom containing C₃-C₂₄ alkyl, substituted or unsubstituted C₂-C₂₄ alkenyl, substituted or unsubstituted heteroatom containing C₂-C₂₄ alkenyl, substituted or unsubstituted C₂-C₂₄ alkynyl, substituted or unsubstituted heteroatom containing C₂-C₂₄ alkynyl, substituted C₅-C₂₄ aryl, unsubstituted C₇-C₂₄ aryl, substituted C₅-C₂₄ heteroaryl, unsubstituted C₇-C₂₄ heteroaryl, substituted or unsubstituted C₁₀-C₂₄ alkaryl, and substituted or unsubstituted heteroatom containing C₁₀-C₂₄ alkaryl.

In some embodiments, any one or more of R¹¹-R¹⁵ comprises a tertiary carbon atom, such as t-butyl, t-butoxy, or analogues thereof.

In some embodiments, R¹¹-R¹⁵ may have van der Waals volumes greater than the van der Waals volume of a trifluoromethyl group, or greater than the van der Waals volume of a trifluoromethoxy group. For example, R¹-R¹⁵ may have van der Waals volumes that are greater than about 70 Å³, or greater than about 80 Å³, or greater than about 90 Å³, or greater than about 100 Å³, or greater than about 120 Å³.

In some embodiments, B is a substituted or unsubstituted alicyclic group. Again, B may be bicyclic or polycyclic, B may comprise one or more substituents and/or one or more heteroatoms, and B may connect to the remainder of the compound at any appropriate location. In the case of bicyclic and polycyclic groups, B may comprise bridging carbon atoms, bridging heteroatoms, fused rings, cyclic substituents, or any combination thereof. Suitable alicyclic groups include substituted and unsubstituted C₆-C₂₄ cycloalkyl, substituted and unsubstituted C₆-C₂₄ heteroatom-containing cycloalkyl, substituted and unsubstituted C₆-C₂₄ cycloalkenyl, and substituted and unsubstituted C₆-C₂₄ heteroatom-containing cycloalkenyl. In addition, B may also be 7- to 12-membered bicyclic and higher order groups such as bicyclo[2.2.1]heptyl, bicyclo[2.2.2]octyl, bicyclo[3.2.1]octayl, bicyclo[3.3.1]nonyl, bicyclo[3.2.2]nonyl, or adamantyl. In some embodiments, B comprises one or more aromatic ring and one or more alicyclic ring.

In some embodiments, B is a secondary or tertiary carbon that has at least two aryl substituents. The aryl substituents may be further substituted with one or more groups selected from halo, alkyl, and halogenated alkyl. For example, B has the structure

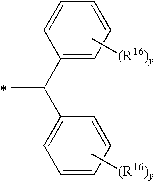

wherein each R¹⁶ is as defined for R¹¹-R¹⁵, and each y is independently selected from 0, 1, 2, 3, 4, and 5.

In some embodiments, B is an aralkyl group. For example, B may be methyl substituted with any of the fused ring systems shown above, such as

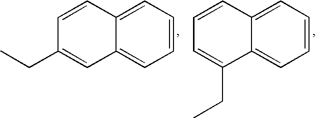

and similarly for other fused ring systems. Again, any of these ring systems may be substituted. Examples of substituents include, for example, halo, hydroxyl, alkyl (including halogenated alkyl), alkoxy (including halogenated alkoxy), aryl, and aryloxy.

Where appropriate, any of the compounds described herein may be administered in the form of a salt, ester, amide, prodrug, conjugate, active metabolite, isomer, fragment, analog, or the like, provided that the salt, ester, amide, prodrug, conjugate, active metabolite, isomer, fragment, or analog is pharmaceutically acceptable and pharmacologically active in the present context. Salts, esters, amides, prodrugs, conjugates, active metabolites, isomers, fragments, and analogs of the agents may be prepared using standard procedures known to those skilled in the art of synthetic organic chemistry and described, for example, by J. March, Advanced Organic Chemistry: Reactions, Mechanisms and Structure, 5th Edition (New York: Wiley-Interscience, 2001).

Any of the compounds of the invention may be the active agent in a formulation as described herein. Formulations containing the compounds of the invention may include 1, 2, or 3 of the compounds described herein, and may also include one or more additional active agents.

The amount of active agent in the formulation typically ranges from about 0.05 wt % to about 95 wt % based on the total weight of the formulation. For example, the amount of active agent may range from about 0.05 wt % to about 50 wt %, or from about 0.1 wt % to about 25 wt %. Alternatively, the amount of active agent in the formulation may be measured so as to achieve a desired dose.

Formulations containing the compounds of the invention may be presented in unit dose form or in multi-dose containers with an optional preservative to increase shelf life.

The compositions of the invention may be administered, e.g. to a patient, by any appropriate method. In general, both systemic and localized methods of administration are acceptable. It will be obvious to those skilled in the art that the selection of a method of administration will be influenced by a number of factors, such as the condition being treated, frequency of administration, dosage level, and the wants and needs of the patient. For example, certain methods may be better suited for rapid delivery of high doses of active agent, while other methods may be better suited for slow, steady delivery of active agent. Examples of methods of administration that are suitable for delivery of the compounds of the invention include parental and transmembrane absorption (including delivery via the digestive and respiratory tracts). Formulations suitable for delivery via these methods are well known in the art.

For example, formulations containing the compounds of the invention may be administered parenterally, such as via intravenous, subcutaneous, intraperitoneal, or intramuscular injection, using bolus injection and/or continuous infusion. Generally, parenteral administration employs liquid formulations.

The compositions may also be administered via the digestive tract, including orally and rectally. Examples of formulations that are appropriate for administration via the digestive tract include tablets, capsules, pastilles, chewing gum, aqueous solutions, and suppositories.

The formulations may also be administered via transmucosal administration. Transmucosal delivery includes delivery via the oral (including buccal and sublingual), nasal, vaginal, and rectal mucosal membranes. Formulations suitable for transmucosal deliver are well known in the art and include tablets, chewing gums, mouthwashes, lozenges, suppositories, gels, creams, liquids, and pastes.

The formulations may also be administered transdermally. Transdermal delivery may be accomplished using, for example, topically applied creams, liquids, pastes, gels and the like as well as what is often referred to as transdermal “patches.”

The formulations may also be administered via the respiratory tract. Pulmonary delivery may be accomplished via oral or nasal inhalation, using aerosols, dry powders, liquid formulations, or the like. Aerosol inhalers and imitation cigarettes are examples of pulmonary dosage forms.

Liquid formulations include solutions, suspensions, and emulsions. For example, solutions may be aqueous solutions of the active agent and may include one or more of propylene glycol, polyethylene glycol, and the like. Aqueous suspensions can be made by dispersing the finely divided active agent in water with viscous material, such as natural or synthetic gums, resins, methylcellulose, sodium carboxymethylcellulose, or other well known suspending agents. Also included are formulations of solid form which are intended to be converted, shortly before use, to liquid form.

Tablets and lozenges may comprise, for example, a flavored base such as compressed lactose, sucrose and acacia or tragacanth and an effective amount of an active agent. Pastilles generally comprise the active agent in an inert base such as gelatin and glycerine or sucrose and acacia. Mouthwashes generally comprise the active agent in a suitable liquid carrier.

For topical administration to the epidermis the chemical compound according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Transdermal patches typically comprise: (1) a impermeable backing layer which may be made up of any of a wide variety of plastics or resins, e.g. aluminized polyester or polyester alone or other impermeable films; and (2) a reservoir layer comprising, for example, a compound of the invention in combination with mineral oil, polyisobutylene, and alcohols gelled with USP hydroxymethylcellulose. As another example, the reservoir layer may comprise acrylic-based polymer adhesives with resinous crosslinking agents which provide for diffusion of the active agent from the reservoir layer to the surface of the skin. The transdermal patch may also have a delivery rate-controlling membrane such as a microporous polypropylene disposed between the reservoir and the skin. Ethylene-vinyl acetate copolymers and other microporous membranes may also be used. Typically, an adhesive layer is provided which may comprise an adhesive formulation such as mineral oil and polyisobutylene combined with the active agent.

Other typical transdermal patches may comprise three layers: (1) an outer layer comprising a laminated polyester film; (2) a middle layer containing a rate-controlling adhesive, a structural non-woven material and the active agent; and (3) a disposable liner that must be removed prior to use. Transdermal delivery systems may also involve incorporation of highly lipid soluble carrier compounds such as dimethyl sulfoxide (DMSO), to facilitate penetration of the skin. Other carrier compounds include lanolin and glycerin.

Rectal or vaginal suppositories comprise, for example, an active agent in combination with glycerin, glycerol monopalmitate, glycerol, monostearate, hydrogenated palm kernel oil and fatty acids. Another example of a suppository formulation includes ascorbyl palmitate, silicon dioxide, white wax, and cocoa butter in combination with an effective amount of an active agent.

Nasal spray formulations may comprise a solution of active agent in physiologic saline or other pharmaceutically suitable carder liquids. Nasal spray compression pumps are also well known in the art and can be calibrated to deliver a predetermined dose of the solution.

Aerosol formulations suitable for pulmonary administration include, for example, formulations wherein the active agent is provided in a pressurized pack with a suitable propellant. Suitable propellants include chlorofluorocarbons (CFCs) such as dichlorodifluoromethane, trichlorofluoromethane, or dichlorotetrafluoroethane, carbon dioxide, or other suitable gases. The aerosol may also contain a surfactant such as lecithin. The dose of drug may be controlled by provision of a metered valve.

Dry powder suitable for pulmonary administration include, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. Unit doses for dry powder formulations may be, for example, in the form of capsules or cartridges of, e.g., gelatin, or blister packs from which the powder may be administered by means of an inhaler.

In addition to the foregoing components, it may be necessary or desirable to incorporate any of a variety of additives, e.g., components that improve drug delivery, shelf-life and patient acceptance. Suitable additives include acids, antioxidants, antimicrobials, buffers, carriers, colorants, crystal growth inhibitors, defoaming agents, diluents, emollients, fillers, flavorings, gelling agents, fragrances, lubricants, propellants, thickeners, salts, solvents, surfactants, other chemical stabilizers, or mixtures thereof. Examples of these additives can be found, for example, in M. Ash and I. Ash, Handbook of Pharmaceutical Additives (Hampshire, England: Gower Publishing, 1995), the contents of which are incorporated herein by reference.

Appropriate dose and regimen schedules will be apparent based on the present invention and on information generally available to the skilled artisan. When the compounds of the invention are used in the treatment of a drug addiction, achievement of the desired effects may require weeks or months of controlled, low-level administration of the formulations described herein.

The amount of active agent in formulations that contain the compounds of the invention may be calculated to achieve a specific dose (i.e., unit weight of active agent per unit weight of patient) of active agent. Furthermore, the treatment regimen may be designed to sustain a predetermined systemic level of active agent. For example, formulations and treatment regimen may be designed to provide an amount of active agent that ranges from about 0.001 mg/kg/day to about 100 mg/kg/day for an adult. As a further example, the amount of active agent may range from about 0.1 mg/kg/day to about 50 mg/kg/day, about 0.1 mg/kg/day to about 25 mg/kg/day, or about 1 mg/kg/day to about 10 mg/kg/day. One of skill in the art will appreciate that dosages may vary depending on a variety of factors, including method and frequency of administration, physical characteristics of the patient, level of drug addiction of the patient, duration of treatment regimen, and the severity of withdrawal symptoms that are experienced by the patient.

Treatment regimens that make use of multiple methods of administration are within the scope of the invention.

The compounds of the invention may be prepared using synthetic methods as exemplified in the experimental section herein, as well as standard procedures that are known to those skilled in the art of synthetic organic chemistry and used for the preparation of analogous compounds. Appropriate synthetic procedures may be found, for example, in J. March, Advanced Organic Chemistry Reactions, Mechanisms and Structure, 5th Edition (New York: Wiley-Interscience, 2001). Syntheses of representative compounds are detailed in the Examples.

A pharmaceutically acceptable salt may be prepared from any pharmaceutically acceptable organic acid or base, any pharmaceutically acceptable inorganic acid or base, or combinations thereof. The acid or base used to prepare the salt may be naturally occurring.

Suitable organic acids for preparing acid addition salts include, e.g., C₁-C₆ alkyl and C₆-C₁₂ aryl carboxylic acids, di-carboxylic acids, and tri-carboxylic acids such as acetic acid, propionic acid, succinic acid, maleic acid, fumaric acid, tartaric acid, glycolic acid, citric acid, pyruvic acid, oxalic acid, malic acid, malonic acid, benzoic acid, cinnamic acid, mandelic acid, salicylic acid, phthalic acid, and terephthalic acid, and aryl and alkyl sulfonic acids such as methanesulfonic acid, ethanesulfonic acid, and p-toluenesulfonic acid, and the like. Suitable inorganic acids for preparing acid addition salts include, e.g., hydrochloric acid, hydrobromic acid, hydroiodic acid, sulfuric acid, nitric acid, and phosphoric acid, and the like. An acid addition salt may be reconverted to the free base by treatment with a suitable base.

Suitable organic bases for preparing basic addition salts include, e.g., primary, secondary and tertiary amines, such as trimethylamine, triethylamine, tripropylamine, N,N-dibenzylethylenediamine, 2-dimethylaminoethanol, ethanolamine, ethylenediamine, glucamine, glucosamine, histidine, and polyamine resins, cyclic amines such as caffeine, N-ethylmorpholine, N-ethylpiperidine, and purine, and salts of amines such as betaine, choline, and procaine, and the like. Suitable inorganic bases for preparing basic addition salts include, e.g., salts derived from sodium, potassium, ammonium, calcium, ferric, ferrous, aluminum, lithium, magnesium, or zinc such as sodium hydroxide, potassium hydroxide, calcium carbonate, sodium carbonate, and potassium carbonate, and the like. A basic addition salt may be reconverted to the free acid by treatment with a suitable acid.

Preparation of Esters Involves Transformation of a Carboxylic Acid Group Via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

The compounds described herein may also be administered in combination therapy regimens with, for example, anti-microbial agents, anti-diabetic agents, analgesics, anti-inflammatory agents, anti-convulsant agents, CNS and respiratory stimulants, neuroleptic agents, hypnotic agents and sedatives, anxiolytics and tranquilizers, other anti-cancer drugs including antineoplastic agents, antihyperlipidemic agents, antihypertensive agents, cardiovascular preparations, anti-viral agents, sex steroids, muscarinic receptor agonists and antagonists, and macromolecular active agents such as peptide drugs.

Other active agents that may be administered in combination with the compounds described herein include, but are not limited to the following:

Anti-microbial agents. These include: tetracycline antibiotics and related compounds (chlortetracycline, oxytetracycline, demeclocycline, methacycline, doxycycline, minocycline, rolitetracycline); macrolide antibiotics such as erythromycin, clarithromycin, and azithromycin; streptogramin antibiotics such as quinupristin and dalfopristin; beta-lactam antibiotics, including penicillins (e.g., penicillin G, penicillin VK), antistaphylococcal penicillins (e.g., cloxacillin, dicloxacillin, nafcillin, and oxacillin), extended spectrum penicillins (e.g., aminopenicillins such as ampicillin and amoxicillin, and the antipseudomonal penicillins such as carbenicillin), and cephalosporins (e.g., cefadroxil, cefepime, cephalexin, cefazolin, cefoxitin, cefotetan, cefuroxime, cefotaxime, ceftazidime, and ceftriaxone), and carbapenems such as imipenem, meropenem and aztreonam; aminoglycoside antibiotics such as streptomycin, gentamicin, tobramycin, amikacin, and neomycin; glycopeptide antibiotics such as teicoplanin; sulfonamide antibiotics such as sulfacetamide, sulfabenzamide, sulfadiazine, sulfadoxine, sulfamerazine, sulfamethazine, sulfamethizole, and sulfamethoxazole; quinolone antibiotics such as ciprofloxacin, nalidixic acid, and ofloxacin; anti-mycobacterials such as isoniazid, rifampin, rifabutin, ethambutol, pyrazinamide, ethionamide, aminosalicylic, and cycloserine; systemic antifungal agents such as itraconazole, ketoconazole, fluconazole, and amphotericin B; antiviral agents such as acyclovir, famcicylovir, ganciclovir, idoxuridine, sorivudine, trifluridine, valacyclovir, vidarabine, didanosine, stavudine, zalcitabine, zidovudine, amantadine, interferon alpha, ribavirin and rimantadine; and miscellaneous antimicrobial agents such as chloramphenicol, spectinomycin, polymyxin B (colistin), bacitracin, nitrofurantoin, methenamine mandelate and methenamine hippurate.

Anti-diabetic agents. These include, by way of example, acetohexamide, chlorpropamide, ciglitazone, gliclazide, glipizide, glucagon, glyburide, miglitol, pioglitazone, tolazamide, tolbutamide, triampterine, and troglitazone.

Analgesics. Non-opioid analgesic agents include apazone, etodolac, difenpiramide, indomethacin, meclofenamate, mefenamic acid, oxaprozin, phenylbutazone, piroxicam, and tolmetin; opioid analgesics include alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol.

Anti-inflammatory agents. Anti-inflammatory agents include the nonsteroidal anti-inflammatory agents, e.g., the propionic acid derivatives as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, and fenbufen; apazone; diclofenac; difenpiramide; diflunisal; etodolac; indomethacin; ketorolac; meclofenamate; nabumetone; phenylbutazone; piroxicam; sulindac; and tolmetin. Steroidal anti-inflammatory agents include hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, and methylprednisolone.

Anti-convulsant agents. Suitable anti-convulsant (anti-seizure) drugs include, by way of example, azetazolamide, carbamazepine, clonazepam, clorazepate, ethosuximide, ethotoin, felbamate, lamotrigine, mephenyloin, mephobarbital, phenyloin, phenobarbital, primidone, trimethadione, vigabatrin, topiramate, and the benzodiazepines. Benzodiazepines, as is well known, are useful for a number of indications, including anxiety, insomnia, and nausea.

CNS and respiratory stimulants. CNS and respiratory stimulants also encompass a number of active agents. These stimulants include, but are not limited to, the following: xanthines such as caffeine and theophylline; amphetamines such as amphetamine, benzphetamine hydrochloride, dextroamphetamine, dextroamphetamine sulfate, levamphetamine, levamphetamine hydrochloride, methamphetamine, and methamphetamine hydrochloride; and miscellaneous stimulants such as methylphenidate, methylphenidate hydrochloride, modafinil, pemoline, sibutramine, and sibutramine hydrochloride.

Neuroleptic agents. Neuroleptic drugs include antidepressant drugs, antimanic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, fluoxetine, fluvoxamine, paroxetine, sertraline, and venlafaxine, (c) monoamine oxidase inhibitors such as phenelzine, tranylcypromine, and (−)-selegiline, and (d) other, “atypical” antidepressants such as nefazodone, trazodone and venlafaxine, and wherein antimanic and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole.

Hypnotic agents and sedatives include clomethiazole, ethinamate, etomidate, glutethimide, meprobamate, methyprylon, zolpidem, and barbiturates (e.g., amobarbital, apropbarbital, butabarbital, butalbital, mephobarbital, methohexital, pentobarbital, phenobarbital, secobarbital, thiopental).

Anxiolytics and tranquilizers include benzodiazepines (e.g., alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, triazolam), buspirone, chlordiazepoxide, and droperidol.

Anticancer agents, including antineoplastic agents: Paclitaxel, docetaxel, camptothecin and its analogues and derivatives (e.g., 9-aminocamptothecin, 9-nitrocamptothecin, 10-hydroxy-camptothecin, irinotecan, topotecan, 20-O-β-glucopyranosyl camptothecin), taxanes (baccatins, cephalomannine and their derivatives), carboplatin, cisplatin, interferon-α_{2A}, interferon-α_{2B}, interferon-α_{N3} and other agents of the interferon family, levamisole, altretamine, cladribine, tretinoin, procarbazine, dacarbazine, gemcitabine, mitotane, asparaginase, porfimer, mesna, amifostine, mitotic inhibitors including podophyllotoxin derivatives such as teniposide and etoposide and vinca alkaloids such as vinorelbine, vincristine and vinblastine.

Antihyperlipidemic agents. Lipid-lowering agents, or “hyperlipidemic” agents, include HMG-CoA reductase inhibitors such as atorvastatin, simvastatin, pravastatin, lovastatin and cerivastatin, and other lipid-lowering agents such as clofibrate, fenofibrate, gemfibrozil and tacrine.

Antihypertensive agents. These include amlodipine, benazepril, darodipine, diltiazem, doxazosin, enalapril, eposartan, esmolol, felodipine, fenoldopam, fosinopril, guanabenz, guanadrel, guanethidine, guanfacine, hydralazine, losartan, metyrosine, minoxidil, nicardipine, nifedipine, nisoldipine, phenoxybenzamine, prazosin, quinapril, reserpine, terazosin, and valsartan.

Cardiovascular preparations. Cardiovascular preparations include, by way of example, angiotensin converting enzyme (ACE) inhibitors, cardiac glycosides, calcium channel blockers, beta-blockers, antiarrhythmics, cardioprotective agents, and angiotensin II receptor blocking agents. Examples of the foregoing classes of drugs include the following: ACE inhibitors such as enalapril, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3 S-1 H-1-benzazepine-1-acetic acid or 3-(1-ethoxycarbonyl-3-phenyl-(1S)-propylamino)-2,3,4,5-tetrahydro-2-oxo-(3S)-benzazepine-1-acetic acid monohydrochloride; cardiac glycosides such as digoxin and digitoxin; inotropes such as aminone and milrinone; calcium channel blockers such as verapamil, nifedipine, nicardipene, felodipine, isradipine, nimodipine, bepridil, amlodipine and diltiazem; beta-blockers such as atenolol, metoprolol; pindolol, propafenone, propranolol, esmolol, sotalol, timolol, and acebutolol; antiarrhythmics such as moricizine, ibutilide, procainamide, quinidine, disopyramide, lidocaine, phenyloin, tocamide, mexiletine, flecamide, encamide, bretylium and amiodarone; and cardioprotective agents such as dexrazoxane and leucovorin; vasodilators such as nitroglycerin; and angiotensin II receptor blocking agents such as losartan, hydrochlorothiazide, irbesartan, candesartan, telmisartan, eposartan, and valsartan.

Other cardiac agents. Examples of other cardiac agents that can be used in combination with the diuretics of the present invention include without limitation: amiodarone, amlodipine, atenolol, bepridil, bisoprolol bretylium, captopril, carvedilol, diltiazem, disopyramide, dofetilide, enalaprilat, enalapril, encamide, esmolol, flecamide, fosinopril, ibutilide, inaminone, irbesartan, lidocaine, lisinopril, losartan, metroprolol, nadolol, nicardipine, nifedipine, procainamide, propafenone, propranolol, quinapril, quinidine, ramipril, trandolapril, and verapamil.

Anti-viral agents. Antiviral agents that can be delivered using the present dosage forms include the antiherpes agents acyclovir, famciclovir, foscarnet, ganciclovir, idoxuridine, sorivudine, trifluridine, valacyclovir, and vidarabine; the antiretroviral agents didanosine, stavudine, zalcitabine, and zidovudine; and other antiviral agents such as amantadine, interferon alpha, ribavirin and rimantadine.

Sex steroids. The sex steroids include, first of all, progestogens such as acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, fluorogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. Also included within this general class are estrogens, e.g.: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. Androgenic agents, also included within the general class of sex steroids, are drugs such as the naturally occurring androgens androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, dehydroepiandrosterone (DHEA; also termed “prasterone”), sodium dehydroepiandrosterone sulfate, 4-dihydrotestosterone (DHT; also termed “stanolone”), 5α-dihydrotestosterone, dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol and testosterone; pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters; and pharmaceutically acceptable derivatives of testosterone such as methyl testosterone, testolactone, oxymetholone and fluoxymesterone.

Muscarinic receptor agonists and antagonists. Muscarinic receptor agonists include, by way of example: choline esters such as acetylcholine, methacholine, carbachol, bethanechol (carbamylmethylcholine), bethanechol chloride, cholinomimetic natural alkaloids and synthetic analogs thereof, including pilocarpine, muscarine, McN-A-343, and oxotremorine. Muscarinic receptor antagonists are generally belladonna alkaloids or semisynthetic or synthetic analogs thereof, such as atropine, scopolamine, homatropine, homatropine methyl bromide, ipratropium, methantheline, methscopolamine and tiotropium.

Peptide drugs. Peptidyl drugs include the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β3-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP), and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin. Still other peptidyl drugs that can be advantageously delivered using the present systems include endorphins (e.g., dermorphin, dynorphin, α-endorphin, β-endorphin, γ-endorphin, α-endorphin, [Leu⁵]enkephalin, [Met⁵]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof.

In some embodiments, provided herein are compounds that display antagonistic effects on PPARδ function. A series of PPARδ antagonists with varying activity profiles are provided that give important insights into the SARS that affect ligand binding and antagonist activity. Where nuclear receptor is over-expressed, as in metastatic human prostate cancers, (see Western data provided herein), PPARδ antagonism is an effective strategy for interfering with PPARδ-dependent processes such as activation of anti-apoptotic pathways, increased cellular proliferation, adhesion, and metastasis.

In some embodiments, the compounds described herein are synthetic antagonists against PPARδ. Thus, the compounds bind to, but do not modulate, PPARδ, and may preferentially bind to PPARδ in place of endogenous or other synthetic PPARδ modulators. Thus, the invention provides a method for reducing or eliminating endogenous modulation of PPARδ or modulation of PPARδ with synthetic PPARδ agonists.

In some embodiments, the compounds described herein are agonists of PPARδ.

The compounds of the invention modulate PPARδ, and may also act in concert with endogenous PPARδ modulators to provide an enhanced response. Thus, the invention provides a method for modulating PPARδ, and further provides a method to enhance modulation of PPARδ by endogenous ligands.

The compounds described herein find utility, for example, in the treatment of certain cancers. A broad range of cancers are treatable with the compounds described herein, including prostate cancer, colorectal carcinoma, breast cancer and non-small cell lung carcinoma. The compounds described herein are, in some embodiments, useful for direct local control of a growing tumor.

Activation of PPARδ by the compounds described herein as well as established PPARδ agonists confers protection against pro-apoptotic stresses in both metastatic prostate and breast cancer cell lines. One of the underlying molecular mechanism involved in this phenotypic change is the activation of a central survival and pro-growth PI3-kinase/Akt/mTOR (rictor) signaling pathway. In addition, adenoviral forced expression of PPARδ in less aggressive prostate cell lines greatly increases signaling through this pathway.

Furthermore, co-incubation of the compounds of the invention (for example, SR 13904) with PPARδ agonists greatly attenuates the effects of activated PPARδ. Signaling through the PI3-kinase pathway is reduced and the protective effect of activated PPARδ on cell survival is reversed.

In the examples that follow, the compounds described herein attenuate molecular and cellular events. It is demonstrated that ligand-activated PPARδ: (a) increases CDK2 levels in post-confluent prostate cancer cells, suggesting possible re-entry into the cell cycle; (b) increases the activity levels of the pro-metastatic MMP-9; (c) activates the anti-apoptotic PI3-kinase/Akt1 signaling pathway; and (d) protects against growth factor withdrawal- and drug-induced apoptosis. Furthermore, there is marked depression of cyclin D1 and CDK2 protein levels in prostate cancer cells treated with a PPARδ antagonist in either agonist- or vehicle-treated cells.

In some embodiments, the invention provides a method for treating a patient suffering form cancer. In some embodiments, the invention provides a method for treating a patient suffering form a condition regulated by PPARδ. In some embodiments, the invention provides a method for modulating the delta subtype of a peroxisome proliferators activated receptor (PPARδ). In some embodiments, the invention provides a method for inhibiting PPARδ-mediated gene expression.

In some embodiments, such methods comprise administering any of the compounds of the invention. For example, the methods comprise administering a compound comprising a substituted thiazole group (e.g., a trisubstituted thiazole group), wherein the thiazole group is substituted with a bulk-providing group.

In some embodiments, the invention provides a compound comprising a thiazole group substituted at the 2-position with a bulk-providing substituent, wherein the compound is capable of functioning as a ligand for PPARδ. In some embodiments, the invention provides a pharmaceutical composition comprising any of the compounds described herein and a pharmaceutically acceptable carrier.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description as well as the examples that follow, are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

### EXAMPLES

The Examples that follow demonstrate the preparation and testing of some representative compounds.

General Testing Procedures. Each ligand was screened for PPARδ binding and transactivation activity through the use of two complementary reporter assays as described below. In both systems, transfection efficiency was standardized by co-transfection of a control Renilla luciferase. U2-OS (human bone cancer line) cells were used for both assays because this is the cell line of choice for screening PPAR ligands. At 18 h post-transfection, GW 501, with and without the ligands of the disclosure, was added to the cells and incubated for an additional 24 h. The cells were then harvested and luciferase activities profiled.

A PPARδ protein ligand-binding domain (LBD) assay. The LBD assay consists of cellular co-transfection of a plasmid containing an LBD/Gal4 fusion construct and a vector containing a UAS-tk-luciferase reporter construct under the transcriptional control of a Gal4 upstream activating sequence. This system was used to test for binding activities of candidate PPARδ ligands to the LBD of PPARδ protein.

A promoter/luciferase reporter assay. This assay involves co-transfection of a PPARδ expression vector and a plasmid containing a luciferase gene under the control of PPARδ responsive DNA element (three copies of a consensus PPAR response element, PPRE). This assay was used to directly test ligand effects on PPARδ-responsive genes.

### Example 1

### Synthetic Procedures and Example Compounds

The compounds shown in Table 1 were prepared as representative examples of the compounds described herein. Synthetic procedures for the preparation of some of the compounds are shown below. Any of the compounds of the invention may be prepared using appropriate variations of the synthetic procedures described herein; the scope and nature of such variations will be apparent to the skilled artisan.

TABLE 1
Representative example compounds

| ID Number | Structure |
|---|---|
| SR 13174 | 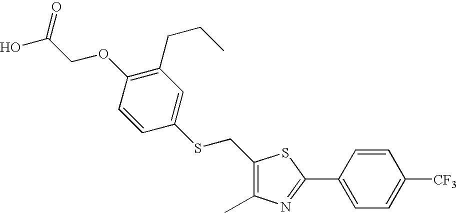 |
| SR 13175 | 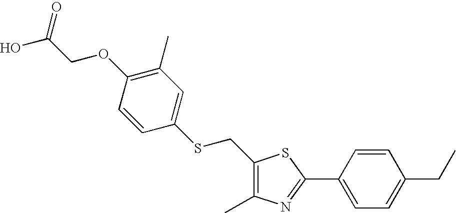 |
| SR 13901 | 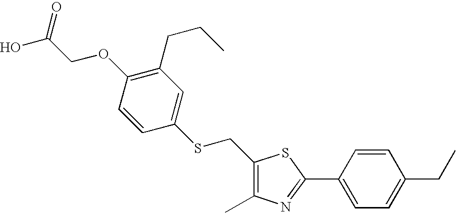 |
| SR 13902 | 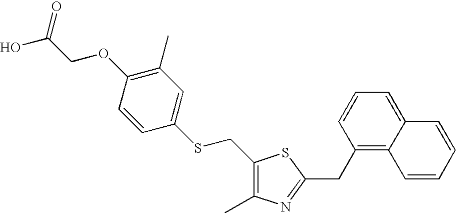 |
| SR 13903 | 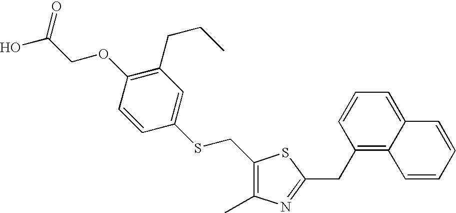 |
| SR 13904 | 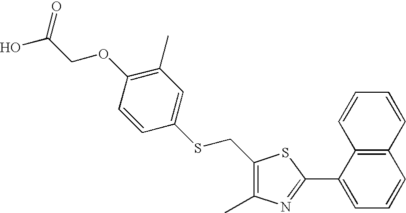 |
| SR 13905 | 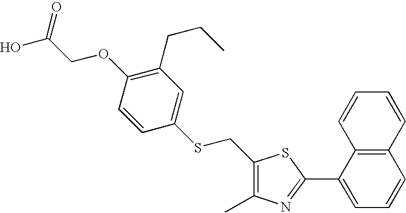 |
| SR 13906 | 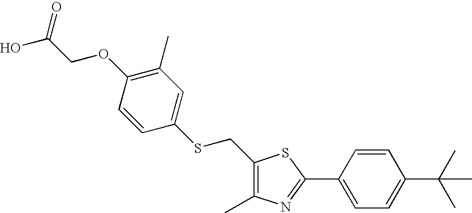 |
| SR 13907 | 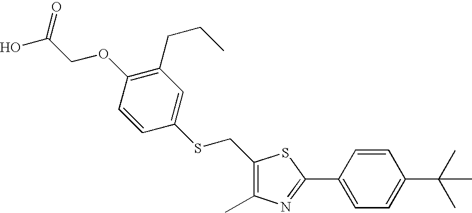 |
| SR 13908 | 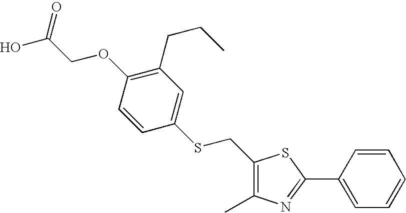 |
| SR 13909 | 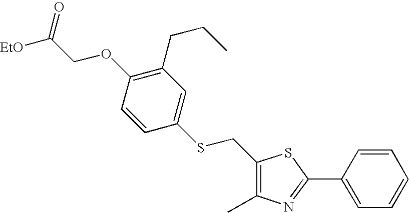 |
| SR 13910 | 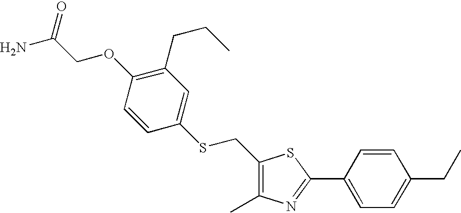 |
| SR 13961 | 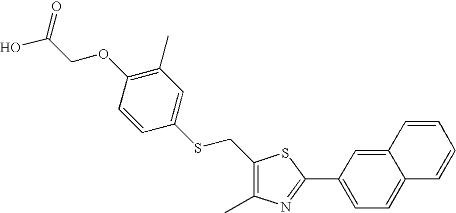 |
| SR 13962 | 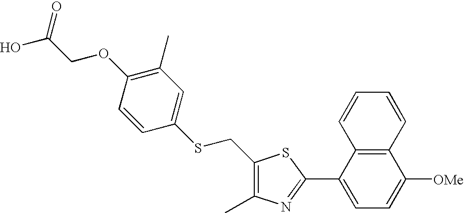 |
| SR 13963 | 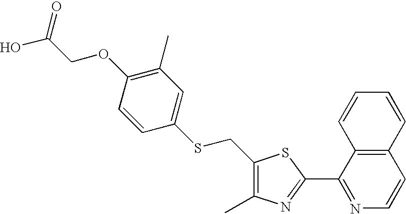 |
| SR 13964 | 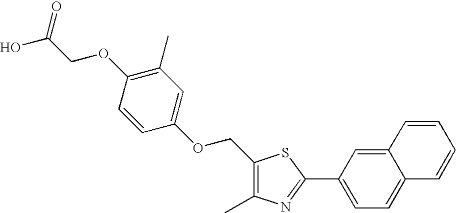 |
| SR 13965 | 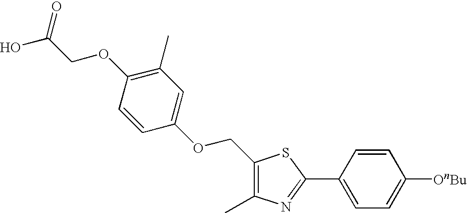 |
| SR 13966 | 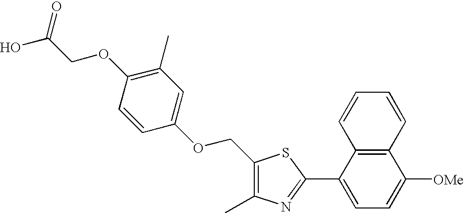 |

Synthesis of PPARδ Ligands SR13904, SR13906.

Scheme 1 shows the step-wise synthetic procedure used to prepare SR13904 and SR13906.

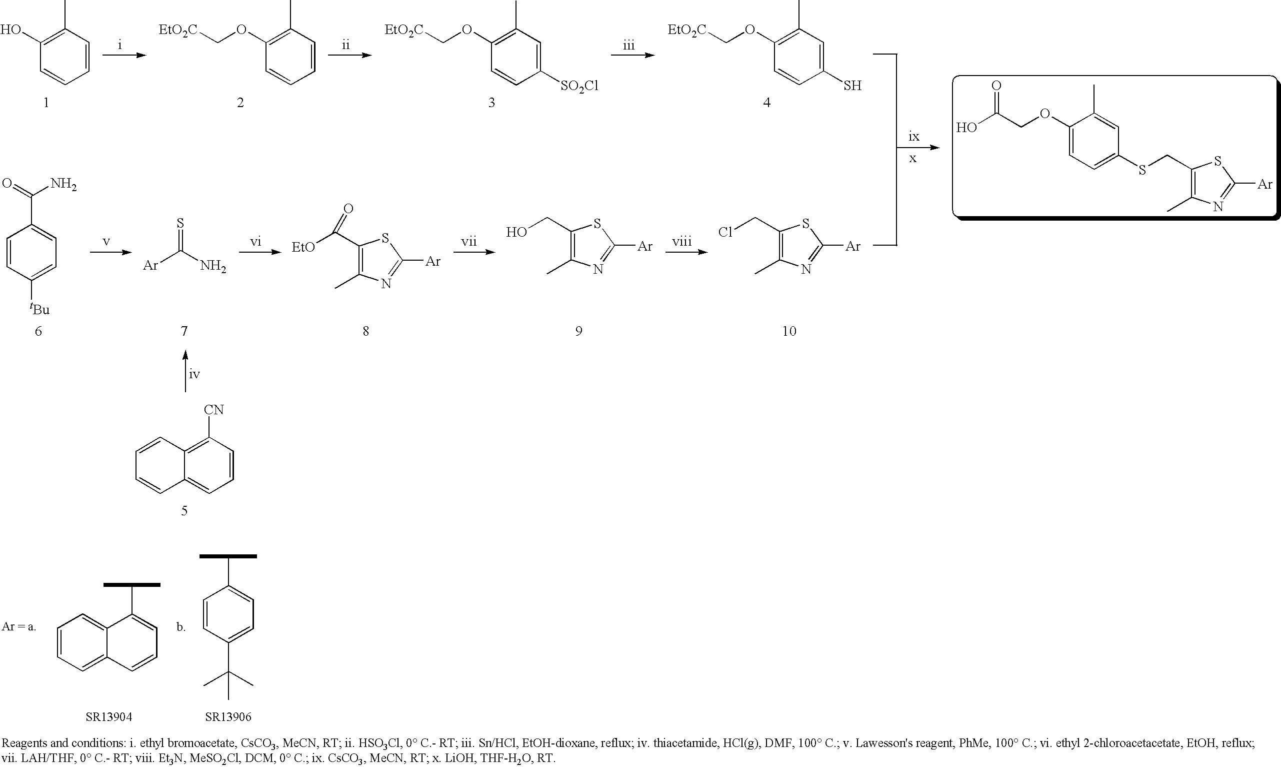

Preparation of 2.

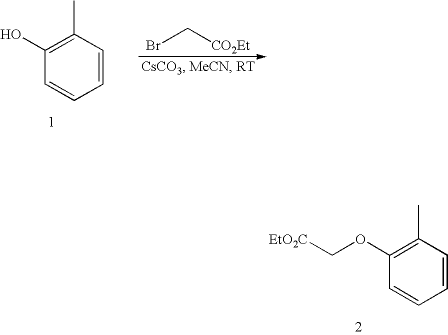

A 1000 mL-RB flask was charged with 2-methylphenol (1) (21.6 g), ethyl bromoacetate (25 mL), Cs₂CO₃ (134 g), and MeCN (500 mL). The mixture was stirred overnight, followed by filtration. The filtrate was evaporated to give an oil; the oil was dissolved in EtOAc (250 mL) and water (80 mL) was added. The mixture was stirred at RT for about 1 h. The organic layer was separated, washed with 1N NaOH and water, dried over sodium sulfate, and evaporated to dryness to afford 40.8 g of crude 2 (˜100%, NMR indicated a small amount of solvent was present). This material was used without further purification in the following step.

¹H NMR (300 MHz, CDCl₃): δ 1.28 (t, J=7.1 Hz, 3H), 2.29 (s, 3H), 4.25 (quart, J=7.1 Hz, 2H), 4.62 (s, 2H), 6.70 (d, J=8.0 Hz, 1H), 6.89 (t, J=8.0 Hz, 1H), 7.08-7.18 (m, 2H).

Preparation of 3.

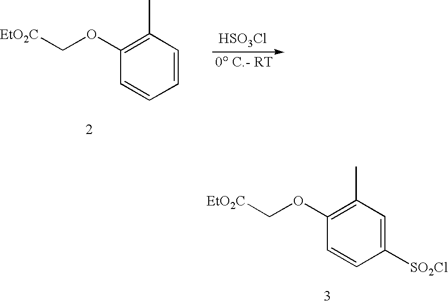

Crude 2 was treated with slow dropwise addition of HSO₃Cl (45 mL), at 0° C. with rapid stirring. After completion of addition, the mixture was stirred at 0° C. for an additional 30 min and then at RT for 2 h, and then poured into ice-water to form a white precipitate. After filtration, the wet solid was dissolved in EtOAc; the aqueous layer was separated, the organic phase dried over sodium sulfate, and evaporated to give 3 as a brown oil (56.5 g, 92%), which solidified while standing at RT. This crude material was used without further purification in the following step. ¹H NMR (300 MHz, CDCl₃): δ 1.32 (t, J=7.2 Hz, 3H), 2.38 (s, 3H), 4.29 (quart, J=7.1 Hz, 2H), 4.76 (s, 2H), 6.81 (d, J=9.4 Hz, 1H), 7.82-7.88 (m, 2H).

Preparation of 4.

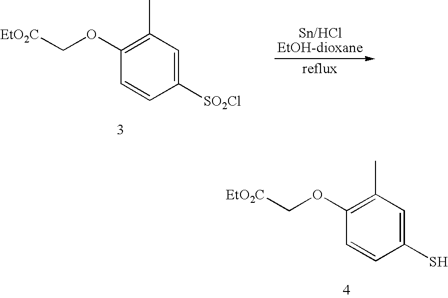

A 100 mL flask was charged with ethyl (4-chlorosulfonyl-2-methylphenoxy)acetate (3) (3.0 g), tin (6.0 g), saturated HCl in dioxane (15 mL), and EtOH (15 mL). After refluxing for 2 h, the solution was poured into ice-water (150 mL) and extracted with DCM (2×120 mL). The extracts were combined, dried over sodium sulfate, and evaporated to a yellow liquid (2.2 g, 98%), which was used without further purification in the coupling step with 10. ¹H NMR (300 MHz, CDCl₃): δ 1.29 (t, J=7.2 Hz, 3H), 2.24 (s, 3H), 4.25 (quart, J=7.2 Hz, 2H), 4.60 (s, 2H), 6.59 (d, J=8.5 Hz, 1H), 6.98-7.18 (m, 2H).

Preparation of 7.

Two methods were applied to make thioamide 7; one started from nitrile 5 (for SR13904) and the other from amide 6 (for SR13906) as indicated in above Scheme I.

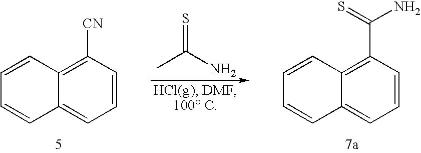

To a solution of 1-cyanonaphthalene 5 (6.2 g) and thioacetamide (7.5 g) in DMF (100 mL), was bubbled HCl gas for 10 min at RT, and the mixture was heated up to 100-105° C. (oil bath) and stirred at this temperature overnight while HCl gas was kept bubbling slowly. The reaction mixture was treated with saturated NaHCO₃ and extracted with EtOAc. The organic extract was washed with brine, dried over sodium sulfate, and evaporated to dark-colored oil, which was subjected to chromatography on silica gel, eluting with EtOAc/hexane (1/4). Starting material 5 (2.3 g) was recovered and 2.1 g of the desired product 7a (44% based on consumed cyanonaphthalene 5) was obtained. ¹H NMR (300 MHz, CDCl₃): δ 7.18 (br, s, 1H), 7.40-7.60 (m, 3H), 7.64 (dd, J=7.2, 1.4 Hz, 1H), 7.83-7.91 (m, 2H), 8.08 (br, s, 1H), 8.28 (dd, J=7.9, 1.2 Hz, 1H); ¹³C NMR (300 MHz, CDCl₃): δ 124.9 (1C), 125.1 (1C), 125.2 (1C), 126.7 (1C), 127.4 (1C), 128.7 (1C), 128.7 (1C), 130.4 (1C), 133.8 (1C), 140.4 (1C), 205.1 (1C).

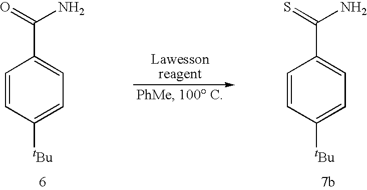

A mixture of 4-tert-butylbenzamide 6 (2.13 g) and Lawesson reagent (3.24 g) in toluene (20 mL) was stirred at 100° C. overnight, followed by evaporation to remove the solvent. The residue was subjected to chromatography on silica gel, eluting with EtOAc/hexane (1/2) to give yellow crystalline product 7b (1.35 g, 58%). ¹H NMR (300 MHz, CDCl₃): δ 1.33 (s, 9H), 7.24 (br, s, 1H), 7.41 (d, J=8.8, 2H), 7.82 (d, 8.8 Hz, 2H), 7.84 (br, s, 1H); ¹³C NMR (300 MHz, CDCl₃): δ 31.3 (3C), 35.2 (1C), 125.7 (2C), 127.1 (2C), 136.5 (1C), 156.1 (1C), 202.7 (1C).

Preparation of 8.

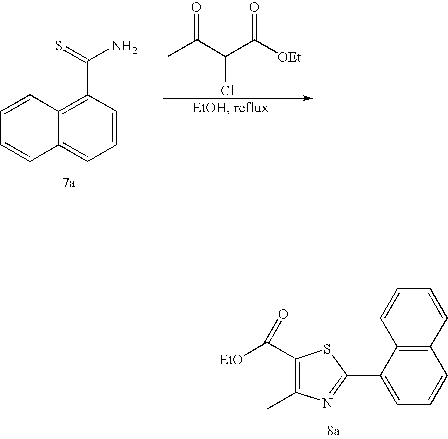

A mixture of 1-naphthalene thiocarboxamide 7a (2.1 g), ethyl 2-chloroacetoacetate (2.0 g), and EtOH (40 mL) was refluxed overnight, followed by removal of the solvent. The residue was partitioned between EtOAc (100 mL) and saturated NaHCO₃ (100 mL); the organic phase was separated, washed with brine, dried over sodium sulfate, and evaporated to an oil, which was subjected to chromatography on silica gel, eluting with EtOAc/hexane (1/5) to give 3.3 g (99%) of 8a as an oil. ¹H NMR (300 MHz, CDCl₃): δ 1.40 (t, J=7.1 Hz, 3H), 2.89 (s, 3H), 4.38 (quat, J=7.1 Hz, 2H), 7.44-8.0 (m, 6H), 8.78 (d, J=8.5, 1H);

¹³C NMR (300 MHz, CDCl₃): δ 14.6 (1C), 17.9 (1C), 61.5 (1C), 122.9 (1C), 125.2 (1C), 125.9 (1C), 126.7 (1C), 127.9 (1C), 128.7 (1C), 129.1 (1C), 130.4 (1C), 130.7 (1C), 131.4 (1C), 134.3 (1C), 160.9 (1C), 162.5 (1C), 169.9 (1C).

Compound 8b was prepared from 7b in the same way in quantitative yield. ¹H NMR (300 MHz, CDCl₃): δ 1.33 (s, 9H), 1.38 (t, J=7.1 Hz, 3H), 2.77 (s, 3H), 4.34 (quat, J=7.0 Hz, 2H), 7.45 (d, J=8.5, 2H), 7.88 (d, J=8.5, 2H); ¹³C NMR (300 MHz, CDCl₃): δ 14.6 (1C), 17.8 (1C), 31.5 (3C), 35.2 (1C), 61.4 (1C), 121.5 (1C), 126.2 (2C), 126.9 (2C), 130.5 (1C), 154.8 (1C), 161.2 (1C), 162.6 (1C), 170.2 (1C).

Preparation of 9.

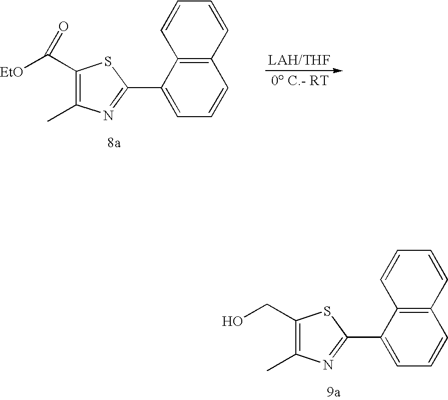

A solution of ethyl 4-methyl-2-(1-naphthyl)-thiazole-5-carboxylate 8a (3.2 g from previous step) in THF (20 mL) was slowly added to an ice-water cooled suspension of LAH (440 mg) in THF (20 mL). After completion of addition, the mixture was stirred at RT for 2 h, and then quenched with water (0.8 mL), and 15% NaOH (0.8 mL). The mixture was stirred at RT for about 30 min, and then filtered; the solid was washed with THF, MeOH, DCM, and EtOAc. All washings were combined with the filtrate, dried over MgSO₄, and evaporated to crude 9a as yellow oil (2.8 g, 100%). This crude product was used without further purification in following step. ¹H NMR (300 MHz, CDCl₃): δ 2.45 (s, 3H), 4.75 (s, 2H), 7.43-7.60 (m, 3H), 7.71 (dt, J=7.0, 1.2 Hz, 1H), 7.84-7.94 (m, 2H), 8.67 (d, J=8.3, 1H).

Compound 9b was prepared from 8b in the same way in quantitative yield. ¹H NMR (300 MHz, CDCl₃): δ 1.32 (s, 9H), 2.36 (s, 3H), 3.14 (br, s, 1H), 4.75 (s, 2H), 7.41 (d, J=8.5, 2H), 7.78 (d, J=8.5, 2H); ¹³C NMR (300 MHz, CDCl₃): δ 15.2 (1C), 31.4 (3C), 35.0 (1C), 56.8 (1C), 126.1 (2C), 126.3 (2C), 131.1 (1C), 131.2 (1C), 150.2 (1C), 153.5 (1C), 166.6 (1C).

Preparation of 10.

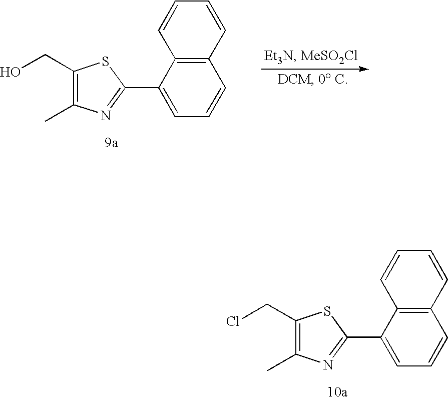

To an ice-water cooled solution of [4-methyl-2-(1-naphthyl)-thiazl-5-yl]-methanol 9a (2.8 g, crude material previous step), triethyl amine (3.1 mL) in DCM (40 mL), was added dropwise methanesulfonyl chloride (1.28 mL). After stirring at 0° C. for 1.5 h, the reaction mixture was added to DCM (50 mL), washed with saturated NaHCO₃ and then brine; the DCM solution was dried over MgSO₄ and then evaporated to crude 10a as a brown oil (2.65 g, 90% in 2 steps). This material was used without further purification in the coupling step with 4. ¹H NMR (300 MHz, CDCl₃): δ 2.57 (s, 3H), 4.83 (s, 2H), 7.45-7.62 (m, 3H), 7.76 (dd, J=7.0, 1.2 Hz, 1H), 7.84-7.94 (m, 2H), 8.76 (d, J=8.1, 1H); ¹³C NMR (300 MHz, CDCl₃): δ 15.4 (1C), 37.6 (1C), 125.3 (1C), 126.0 (1C), 126.6 (1C), 127.6 (1C), 128.6 (1C), 128.6 (1C), 128.7 (1C), 130.7 (1C), 130.8 (2C), 134.2 (1C), 152.7 (1C), 166.6 (1C).

Compound 10b was prepared from 9b in the same way in a yield greater than 90%. ¹H NMR (300 MHz, CDCl₃): δ 1.33 (s, 9H), 2.46 (s, 3H), 4.77 (s, 2H), 7.43 (d, J=8.5, 2H), 7.81 (d, J=8.5, 2H); ¹³C NMR (300 MHz, CDCl₃): δ 15.3 (1C), 31.4 (3C), 35.1 (1C), 37.9 (1C), 126.1 (2C), 126.4 (2C), 127.3 (1C), 130.9 (1C), 152.8 (1C), 153.9 (1C), 167.3 (1C).

Preparation of SR13904 and SR13906.

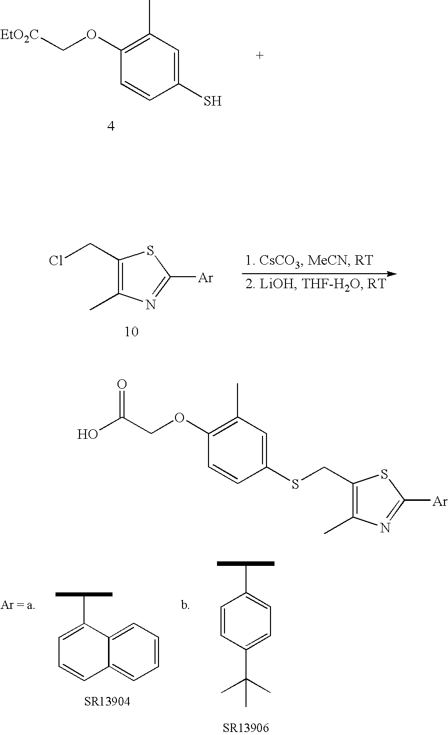

A mixture of ethyl (4-mercapto-2-methylphenoxy)acetate 4 (2.2 g), 5-chloromethyl-4-methyl-2-(1-naphthyl)-thiazole 10a (2.65 g), Cs₂CO₃ (8.0 g), and acetonitrile (50 mL) was stirred at RT overnight, followed by addition of EtOAc (100 mL) and water (50 mL). The organic layer was separated, washed with 1N NaOH and brine, dried over sodium sulfate, and evaporated to give sticky oil (4.5 g). The oil was dissolved in THF (100 mL) and 1N LiOH (20 mL) was added to the THF solution. The resulted mixture was stirred at RT for 24 h and then neutralized with 1N HCl (30 mL) and diluted with EA (100 mL). The organic layer was separated, washed with water, dried over sodium sulfate, and evaporated to give an oil, which subjected to chromatography on a short silica gel column, eluting with DCM/MeOH (100/2) to give 2.9 g (69%) of SR13904 as semisolid. ¹H NMR (300 MHz, CDCl₃): δ 2.10 (s, 3H), 2.10 (s, 3H), 4.11 (s, 2H), 4.52 (s, 2H), 6.51 (d, J=8.4, 1H), 7.10 (dd, J=8.1, 2.1 Hz, 1H), 7.21 (d, J=2.4, 1H), 7.43-7.59 (m, 3H), 7.70 (dd, J=7.2, 1.2 Hz, 1H), 7.84-7.96 (m, 2H), 8.49 (d, J=8.0, 1H), 9.95 (br, s, 1H); ¹³C NMR (300 MHz, CDCl₃): δ 14.2 (1C), 16.1 (1C), 32.2 (1C), 65.2 (1C), 111.5 (1C), 124.9 (1C), 124.9 (1C), 125.4 (1C), 126.3 (1C), 127.2 (1C), 128.2 (1C), 128.3 (1C), 128.4 (1C), 130.1 (1C), 130.4 (1C), 130.4 (1C), 130.5 (1C), 132.4 (1C), 133.8 (1C), 136.4 (1C), 150.0 (1C), 156.3 (1C), 165.0 (1C), 172.0 (1C).

Compound SR13906 was prepared from coupling of 4 and 10b in the same way in a yield of 81%. ¹H NMR (300 MHz, CDCl₃): δ 1.34 (s, 9H), 2.09 (s, 3H), 2.22 (s, 3H), 4.05 (s, 2H), 4.63 (s, 2H), 6.61 (d, J=8.2, 1H), 7.07 (d, J=8.2, 1H), 7.20 (s, 1H), 7.42 (d, J=7.8, 2H), 7.76 (d, J=8.1, 2H), 10.58 (br, s, 1H); ¹³C NMR (300 MHz, CDCl₃): δ 16.3 (1C), 31.4 (3C), 32.7 (1C), 35.1 (1C), 65.7 (1C), 111.8 (1C), 125.3 (1C), 126.2 (2C), 126.6 (2C), 128.6 (1C), 129.4 (1C), 130.3 (1C), 132.6 (1C), 136.5 (1C), 150.3 (1C), 154.0 (1C), 156.7 (1C), 166.7 (1C), 172.3 (1C).

Synthesis of PPARδ Ligands SR13964, SR13966.

Scheme 2 shows the step-wise synthetic procedures that may be used to prepare SR13964 and SR13966.

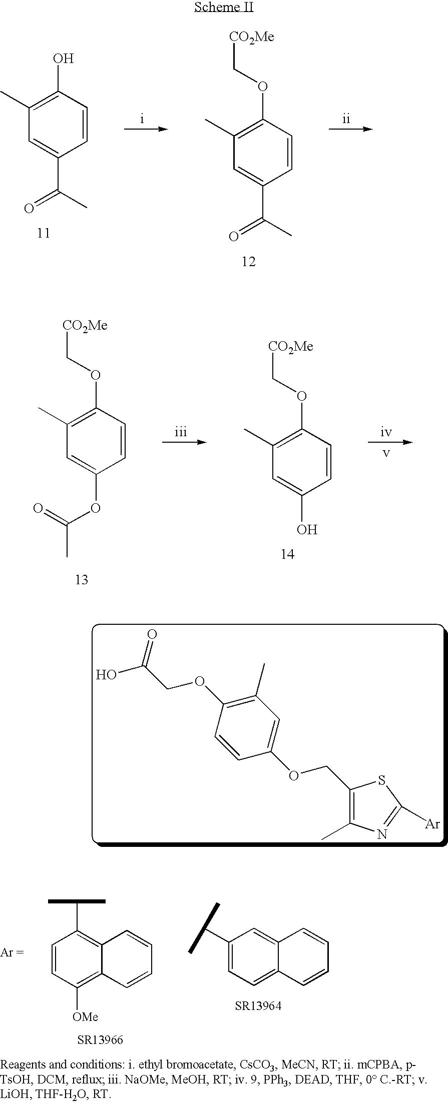

Preparation of 12.

A mixture of 4′-hydroxy-3′-methylacetophenone (11) (5.14 g), methyl bromoacetate (6.00 g), Cs₂CO₃ (22.0 g), and MeCN (160 mL) was stirred at RT overnight; TLC (EtOAc/hexane:1/5) indicated only one spot (R_{f}: 0.26). The mixture was filtered, the solid washed with acetonitrile, and the combined acetonitrile solution evaporated to give a syrup. The residue was dissolved in EtOAc (400 mL), washed with 1N NaOH and water, dried over sodium sulfate, and evaporated to dryness to afford crude 12, a syrup (7.21 g, 95%). This material was used without further purification in the following step. ¹H NMR (300 MHz, CDCl₃): δ 2.30 (s, 3H), 2.51 (s, 3H), 3.78 (s, 3H), 4.71 (s, 2H), 6.70 (d, J=8.5 Hz, 1H), 7.70-7.80 (m, 2H); ¹³C NMR (300 MHz, CDCl₃): δ 16.3 (1C), 26.4 (1C), 52.3 (1C), 65.3 (1C), 110.3 (1C), 127.4 (1C), 128.3 (1C), 130.9 (1C), 131.4 (1C), 160.0 (1C), 169.0 (1C), 196.9 (1C).

Preparation of 13.

A mixture of 12 (1.18 g), mCPBA (1.48 g), p-toluenesulfonic acid (120 mg), and DCM (30 mL) was refluxed for 3.5 h; TLC (EtOAc/hexane:1/1) indicated no starting material and only one spot (R_{f}: 0.83) after this period. The solvent was removed and the residue dissolved in EtOAc (50 mL), washed successively with saturated KI (2×100 mL), NaHSO₃ (2×100 mL), water (2×100 mL), dried over sodium sulfate, and evaporated to afford a brown liquid, crude 13, a syrup (1.26 g, 100%). NMR and TLC indicated only very small amounts of impurities present. This material was used without further purification in the following step. ¹H NMR (300 MHz, CDCl₃): δ 2.24 (s, 3H), 2.26 (s, 3H), 3.77 (s, 3H), 4.61 (s, 2H), 6.67 (d, J=8.7 Hz, 1H), 6.82 (dd, J=8.8, 2.8 Hz, 1H), 6.88 (d, J=2.7 Hz, 1H); ¹³C NMR (300 MHz, CDCl₃): δ 16.5 (1C), 21.2 (1C), 52.4 (1C), 66.2 (1C), 112.1 (1C), 119.4 (1C), 124.2 (1C), 128.9 (1C), 144.9 (1C), 154.0 (1C), 169.7 (1C), 170.1 (1C).

Preparation of 14.

A mixture of 13 (1.50 g), NaOMe (0.50 g), and MeOH (60 mL) was stirred at RT for 0.5 h; TLC (EtOAc/hexane: 1/1) indicated no starting material and only one spot (R_{f}: 0.66). The solvent was removed under vacuum, and the residue neutralized with 1N HCl and extracted with EtOAc (50 mL). The extract was washed with water, dried over sodium sulfate, and evaporated to give crude 14, a brown solid (1.16 g, 94%). NMR and TLC indicated only very small amounts of impurities present. This material was used without further purification in the following step. ¹H NMR (300 MHz, CDCl₃): δ 2.20 (s, 3H), 3.79 (s, 3H), 4.57 (s, 2H), 6.50-6.66 (m, 3H); ¹³C NMR (300 MHz, CDCl₃): δ 16.4 (1C), 52.5 (1C), 66.9 (1C), 113.0 (1C), 113.5 (1C), 118.4 (1C), 129.2 (1C), 150.4 (1C), 150.5 (1C), 170.7 (1C).

Preparation of SR13966.

To an ice-water cooled mixture of 14 (196 mg), [2-(4-methoxy-1-naphthyl)-4-methyl-thiazl-5-yl]-methanol [9 (Ar=4-methoxy-1-naphthyl)] (240 mg), PPh₃ (318 mg), and THF (12 mL), was added dropwise, with efficient stirring, diethylazodicarboxylate (DEAD, 200 μL) over 5 min. The mixture was stirred at RT for 2 days. The solvent was removed and the reside subjected to chromatography on silica gel, eluting with DCM; a fraction (R_{f}: 0.34) was collected to give a yellow solid (108 mg, 28%). The solid was dissolved in THF (10 mL) and 1N LiOH (1.0 mL) was added to the THF solution. The resulted mixture was stirred at RT overnight and then neutralized with 1N HCl and extracted with EtOAc. The extract was washed with brine, dried over sodium sulfate, and evaporated to give SR13966, a solid (99 mg, 95%). TLC indicated only one spot present (R_{f}: 0.29, DCM/MeOH:100/15). ¹H NMR (300 MHz, DMSO): δ 2.19 (s, 3H), 2.47 (s, 3H), 3.39 (br, 1H), 3.99 (s, 3H), 4.62 (s, 2H), 5.21 (s, 2H), 6.76-6.84 (m, 1H), 6.89 (d, J=2.7 Hz, 1H), 7.00 (d, J=8.7 Hz, 1H), 7.51-7.61 (m, 2H), 7.78 (d, J=8.1, 1H), 8.24 (d, J=8.4 Hz, 1H), 8.91 (d, J=8.4, 1H); ¹³C NMR (300 MHz, DMSO): δ 15.9 (1C), 16.9 (1C), 56.5 (1C), 62.7 (1C), 66.1 (1C), 104.7 (1C), 113.0 (1C), 113.1 (1C), 118.7 (1C), 122.5 (1C), 123.2 (1C), 125.8 (1C), 126.2 (1C), 126.5 (1C), 127.8 (1C), 128.1 (1C), 128.5 (1C), 130.0 (1C), 131.2 (1C), 151.3 (1C), 152.6 (1C), 157.0 (1C), 166.0 (1C), 171.2 (1C). MS (ESI(−)): 448 (M−1).

Preparation of SR13964.

The same procedure as above was used in this preparation, starting from 14 (196 mg), [4-methyl-2-(2-naphthyl)-thiazl-5-yl]-methanol [9 (Ar=2-naphthyl)] (228 mg), PPh₃ (320 mg), and diethylazodicarboxylate (DEAD, 200 μL), and resulting in 178 mg (46%) of methyl ester of SR13964 as a yellow solid after chromatography (R_{f}: 0.43, DCM), and SR13964 (163 mg, 95%) obtained after hydrolysis of the ester. ¹H NMR (300 MHz, DMSO): δ 2.19 (s, 3H), 2.42 (s, 3H), 3.41 (br, 1H), 4.63 (s, 2H), 5.17 (s, 2H), 6.77 (s, 2H), 6.87 (s, 1H), 7.48-7.56 (m, 2H), 7.84-8.06 (m, 4H), 8.44 (s, 1H); ¹³C NMR (300 MHz, DMSO): δ 15.8 (1C), 16.9 (1C), 62.8 (1C), 66.1 (1C), 113.0 (1C), 113.2 (1C), 118.7 (1C), 124.1 (1C), 125.9 (1C), 127.6 (1C), 127.8 (1C), 128.1 (1C), 128.4 (1C), 128.5 (1C), 129.3 (1C), 129.4 (1C), 131.1 (1C), 133.6 (1C), 134.3 (1C), 151.3 (1C), 151.9 (1C), 152.5 (1C), 165.8 (1C), 171.2 (1C). MS (ESI(−)): 418 (M−1).

### Example 2

### Activity of Selected Ligands

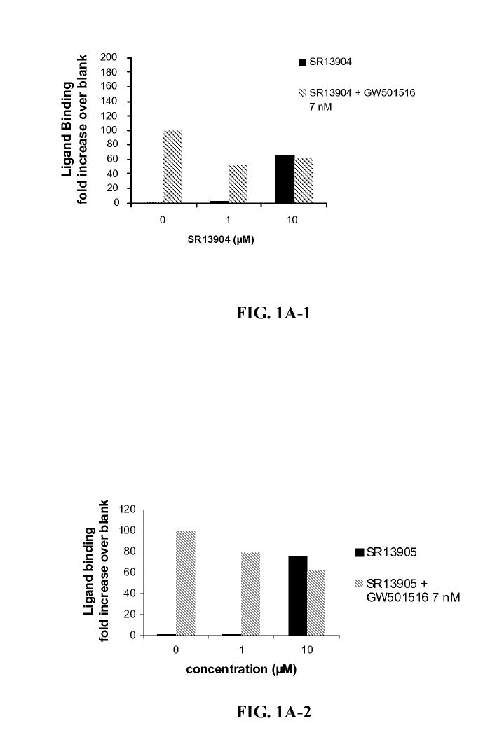

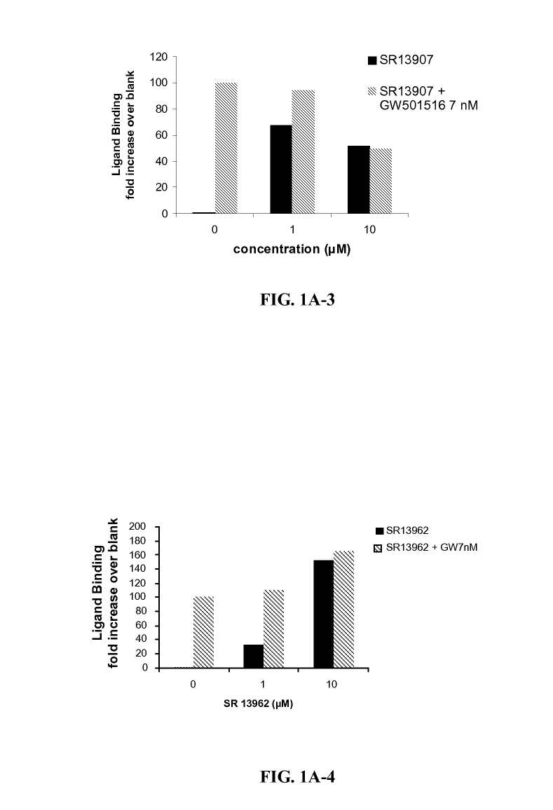

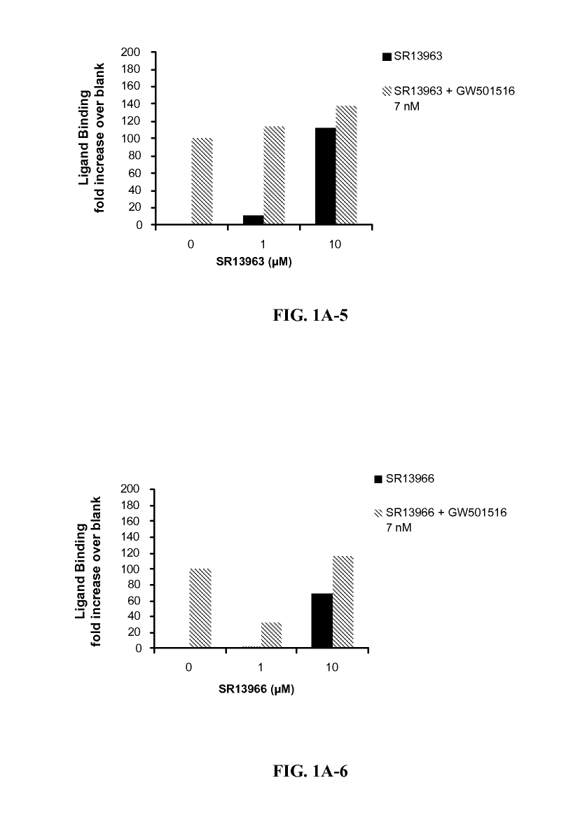

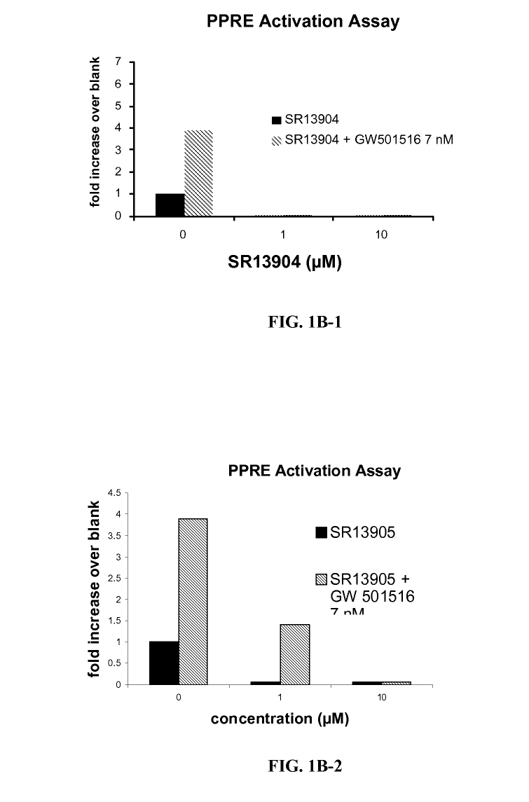

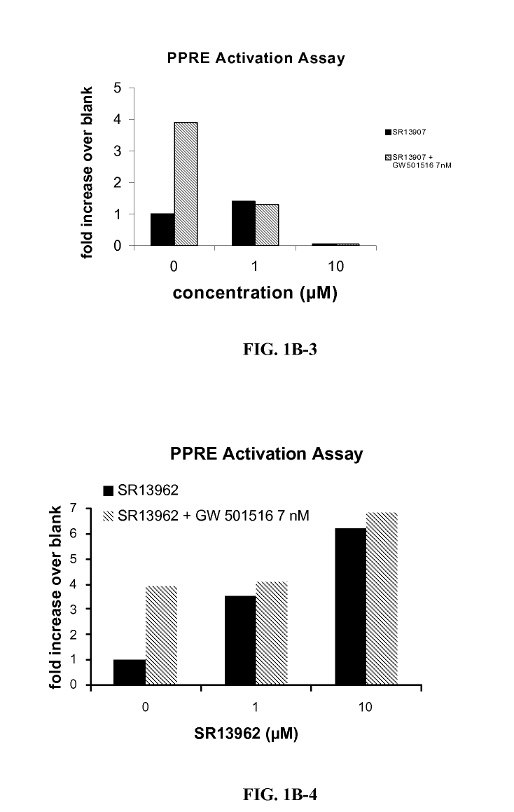

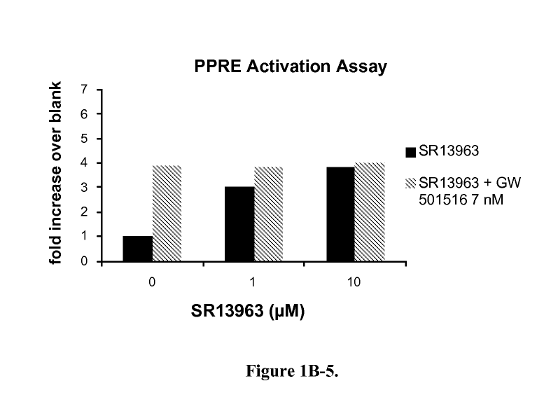

FIG. 1 shows PPARδ antagonist activity of selected ligands of the invention. SR 13904 binds to the LBD of PPARδ, at concentrations in the low micromolar range (1-10 μM), effectively attenuating LBD binding of GW501 (7 nM). This competition of SR 13904 with GW501 was reflected in significantly reduced Gal-4-controlled UAS-tk luciferase activities when cells were treated with both ligands as compared to GW 501 alone. The transactivation assay confirmed this antagonist behavior of SR 13904 toward PPARδ agonists. Both levels (1 and 10 μM) of SR 13904 completely eliminated the transcription activation of the PPRE reporter to GW501.

SR 13904 was also shown to interfere with ciglitazone binding to PPARγ LBD and transactivation (data not shown). Based on the cellular and molecular data generated, treatment of prostate cancer cells with SR 13904 attenuates a number of pro-tumorigenic activities that are likely to be associated with PPARδ. Furthermore, this nuclear receptor is now believed to be a master regulator of nuclear receptors, including both PPARγ and PPARα, and as such the inhibition of PPARδ activity modulates the other target transcription factors.

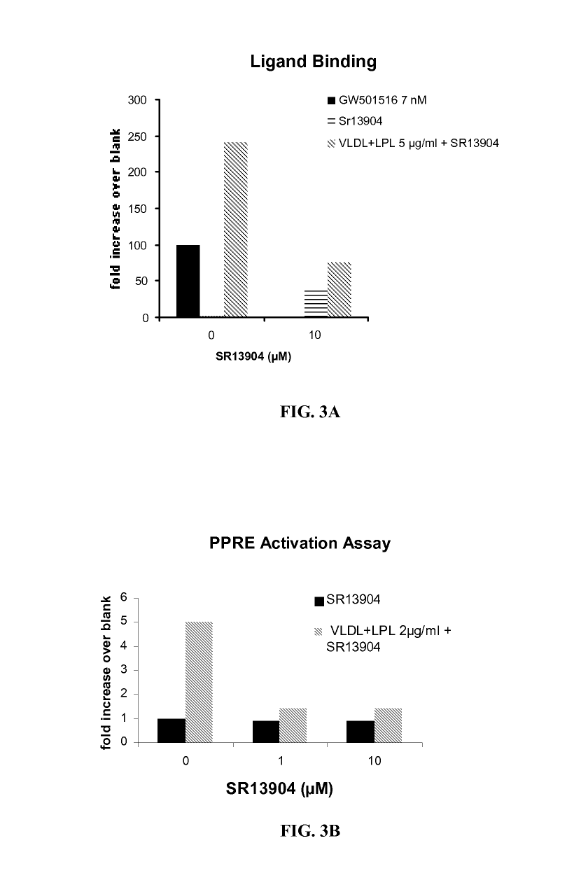

Structure-activity relationships in the new series of PPARδ ligands. All newly synthesized ligands were screened for PPARδ binding by the GAL-4 reporter assay and for PPARδ transactivation in the PPRE reporter assay. Several interesting structure-activity relationship (SAR) trends were observed. Results showed that modifications of the aryl thiazole portion of the lead PPARδ agonist, GW 501, resulted in changes in profile from an agonist to an antagonist. For instance, replacement of the 4-trifluoromethyl-phenyl group on the thiazole ring of GW 501 with a 1-naphthyl group, as in SR 13904, produced a PPARδ antagonist. SR 13961, SR 13906, SR 13907, and SR 13910 were found to be low-affinity agonists. The binding and transactivation profiles of SR 13904 are shown in FIG. 1. Although their ligand binding affinity and luciferasae activation, when each compound was used alone, were lower than that of 7 nM GW 501, both appeared to produce luciferase activation when used in combination with GW 501. This result indicates that SR 13906 and SR 13907 bind at the same site as GW 501 and compete with GW binding. However, in the PPRE transactivation assay (FIG. 3), SR 13904, at 1 μM, significantly decreased the reporter expression via PPRE when used in combination with GW 501, and at 10 μM, it completely inhibited reporter expression. This finding indicates that although SR 13904 has affinity for the receptor, it reduces PPRE reporter expression and PPARδ mediated transactivation, and thus, functionally, it should inhibit PPARδ-mediated gene expression. SR 13905 also has a similar profile to SR 13904. Thus, the ligands of the invention include several that appear to inhibit PARδ-mediated transactivation. Among these are SR 13904, SR 13905, SR 13906, SR 13907, and SR 13967.

### Example 3

### In vitro Evaluation of PPARδ Antagonists

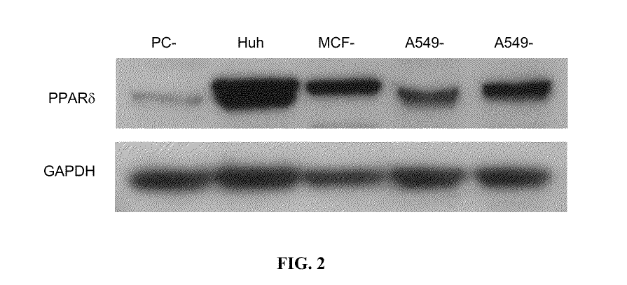

Referring now to FIG. 2, PPARd expression in tumor cell lines was studied. Cultures of A549, Huh7, MCF-7, and PC-3 lines were grown to near confluency (70-80%), lysed, total cellular protein extracted, blotted, and probed for PPARd and GAPDH (control) proteins. The A549 cultures were grown at two densities: the A549-a lane represents the NSCLC line harvested at 70-80% confluency and A549-b at postconfluent growth.

SR13904 attenuates proliferation and colony formation of human cancer cells. Lung (A549), liver (Huh7), prostate (PC-3), and breast (MCF-7) carcinomas were selected to determine the effect of SR13904 on proliferation and colony formation. Western blotting showed that all four-cell lines express significant levels of the PPARδ protein (FIG. 2).

The effect of SR13904 on the proliferation of these cell lines was determined by an Alamar Blue assay. Exponentially growing cells were treated with a range of SR13904 concentrations (0-40 μM; in 0.5% serum) over 4 days and proliferation curves were determined. The IC₅₀ values for growth inhibition by SR13904 for each cell line were determined. The A549 and Huh7 lines displayed the highest sensitivity (IC₅₀: 8-10 μM) and the PC-3 cells the lowest sensitivity (>30 μM) to SR13904. In addition, colony formation assays with SR13904 were performed with both A549 and Huh7 cells. The IC₅₀ values for SR13904 in colony formation assays were found to be in concentration ranges similar to those seen for proliferation (data not shown).

Very low-density lioprotein acts as a potent PPARδ agonist. To examine the molecular consequences of PPARδ inhibition by SR 13904, the triglyceride-rich very low density lipoprotein (VLDL) particle was used as an agonist. VLDL was confirmed as a specific PPARδ agonist using the established PPARδ LBD/Gal4 (and PPARγ LBD/Gal4) and the PPRE reporter assays described above. At the relative concentrations used, VLDL binds to the PPARδ LBD with over twofold higher binding than that seen for GW501. It was also observed that SR13904 does significantly inhibit the binding of VLDL to PPARδ. FIG. 3B indicates that binding of VLDL to the PPARδ LBD leads directly to transactivation of a PPRE reporter. Co-treatment of cells with 1 μM of SR13904 is sufficient to completely block this VLDL-related activation of the PPAR promoter element construct.

Referring to FIG. 3, U2-OS cells were transiently transfected with appropriate expression and reporter vectors. Ligands were added to the cell cultures 24 h post-transfection and incubated for an additional 16 h. Lipoprotein lipase (LPL; 2 μg/ml) was added with VLDL (5 μg/ml) to release PPARδ-active triglycerides. FIG. 3A: PPARδ LBD/Gal4/UAS-tk-luc competition assays with VLDL SR13904 (0-10 μM). The reporter activation (fold increase over blank) is presented relative to GW501516 at 7 nM (fold increase over blank=100). FIG. 3B: PPRE reporter assays using 0, 1, and 10 μM SR13904+VLDL. All assays were repeated in triplicate.

### Example 4

### SR13904 Antagonizes VLDL-Dependent Increases in G1 Cell Cycle Proteins in Post-Confluent Prostate Cancer (PC-3) Cells

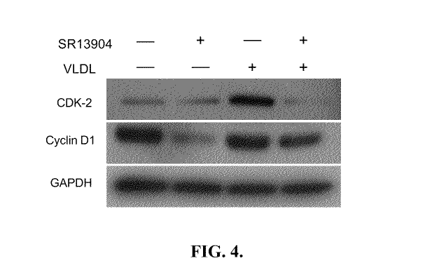

PPARδ plays a role in post-confluent proliferation of 3T3C2 and vascular smooth muscle cells. Although PPARδ activation was found to have little effect on exponential cellular growth of prostate cancer cells, FIG. 4 shows that activation of PPARδ by VLDL resulted in increased CDK2 protein levels in a post-confluent PC-3 prostate cancer cell line.

Conversely, SR 13904 decreased the levels of both CDK2 and cyclin D1 in both vehicle- and VLDL-treated cells suggesting endogenously active PPARδ. These data suggest that PPARδ activation can push quiescent prostate cancer cells back into the cell cycle. The data also support that PPARδ agonists decrease cyclin D1 levels in MCF-7 cells through induction of proteasome-dependent degradation of the protein. Other studies showed that the anticancer effects of PPARδ are inhibited by the cancer-causing cyclin D1. The cyclin D1 gene is amplified or overexpressed in about 50% of human cancers, and this amplification correlates with tumor metastasis, including metastasis of prostate cancer to bone. Another study suggests that the inhibition of PPARδ by cyclin D1 represents a newly discovered mechanism of signal transduction cross-talk between PPARδ ligands and mitogenic signals that induce cyclin D1. The PPARδ studies offer new prototypic therapeutic agents (e.g., SR 13904) to inhibit PPARδ signaling and thus down-regulate cyclin D1 expression. A combination therapy of PPARδ inhibition and PPARδ activation may more effectively modulate tumor cyclin D1 content than therapy with PPARδ agonists alone.

Referring to FIG. 4, PC-3 cells were brought to confluence and cultured in serum-free OptiMem culture medium (Gibco) 2 h before treatment with a combination of VLDL (2 μg/ml+2 μg/ml LPL) and/or SR 13904 (10 μM) and incubated for an additional 48 h. Standard Western blotting was performed. Glyceraldehyde 3 phosphate dehydrogenase (GAPDH) was used a control for both blots.

### Example 5

### Inhibitory Effects on the Cell Cycle

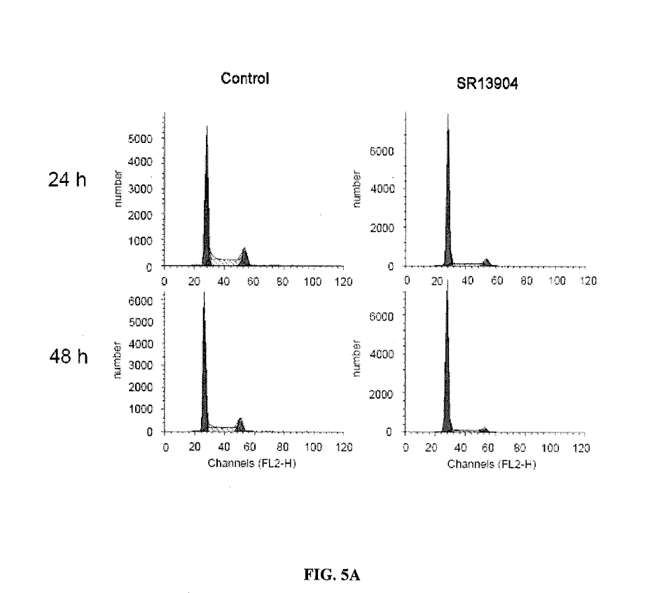

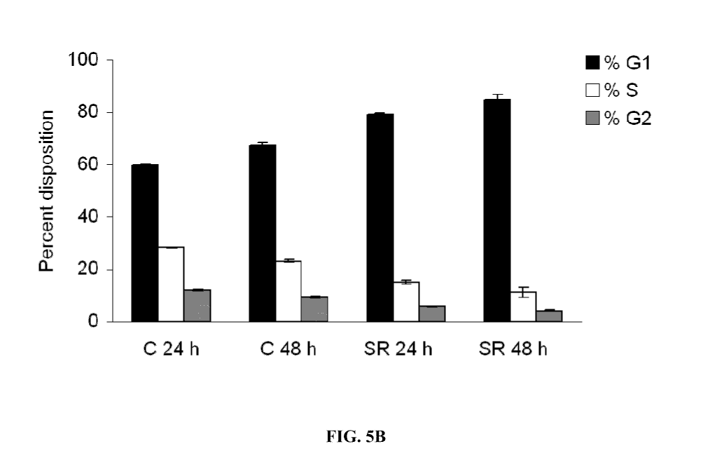

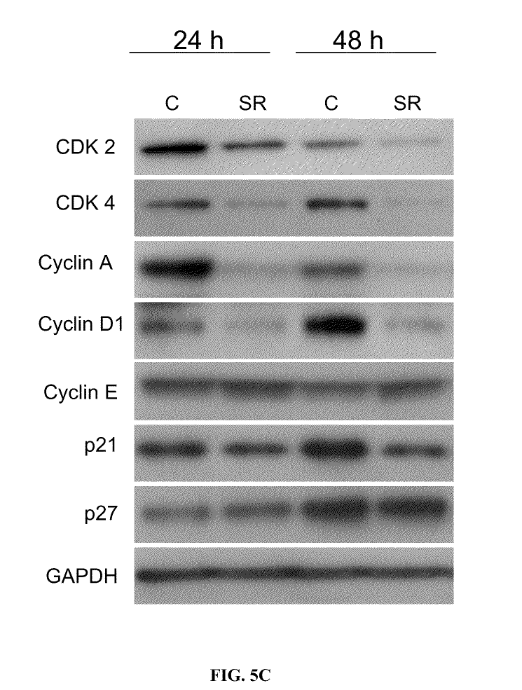

Referring now to FIG. 5A, SR13904 exerts inhibitory effects on the cell cycle. A representative experiment in which A549 cells were grown in phenol-red-free DMEM+2% charcoal-treated FBS □ SR13904 (20 mM). Cells were treated in the exponential phase and assessed by flow cytometry at 24 and 48 h.

Referring now to FIG. 5B, SR13904 treatment arrests A549 cells in the G1 phase of the cell cycle. A549 cells were incubated in 2% FBS (to maintain a high proliferative state) and the cell-cycle profile was determined by flow cytometry after SR13904 treatment (15 μM) for 24 and 48 h, and compared with similar treatment by a control. FIG. 5B shows that SR13904 significantly increased the fraction of cells in the G1 phase and decreased that in the S phase after 24 h, indicating a G1 block. Statistical analysis showed that the differences in cell cycle distribution between control and SR13904-treated cells were highly significant. All differences (SR13904-treated vs. vehicle control-treated) were significant; the G1 differences were highly significant (**p=0.001-0.01, ***p=<0.001). The results provide confirming evidence that activated PPARδ increases cell cycle progression through a positive effect on G1-phase cells, and that PPARδ activation upregulates CDK2, CDK4, and cyclin D1 expression patterns.

Referring now to FIG. 5C, SR13904 treatment of A549 human NSCLC cell line modulates cell cycle protein expression patterns toward an inhibitory phenotype. Given the flow cytometry results described above, the effect of SR13904 on the expression of some key cell-cycle regulatory proteins involved in G1 and S phases was investigated. A549 cells were grown in phenol-red-free DMEM+0.5% charcoal-treated FBS and exposed to the ligand. Relative protein levels were assessed at 24 h and 48 h following addition of SR13904 (20 mM) or control. Western blotting of A549 cells (at 24 and 48 h) showed that SR13904 (20 μM) treatment decreased the steady state levels of CDK2, CDK4, cyclin D1, and cyclin A proteins while having little or no effect on the levels of cyclin E, p21, and p27. These results are generally consistent with other studies that found CDK2 and cyclin D1 protein levels to be increased by treatment with either GW501 or VLDL in MCF-7 and PC-3 cells and this effect to be reversed by adding SR13904, either alone or with the PPARδ agonist (data not shown).

Referring now to FIG. 5D, to better define the level of regulation of SR13904 on cell cycle proteins, CDK2, CDK4, and cyclin D1 were selected for mRNA analysis using real time PCR. A549 cultures were treated with SR13904 (20 μM; 0.5% FBS) and sampled at 24- and 48-h time-points. SR13904 significantly reduced the steady state levels of CDK2 (graph A in FIG. 5D) but had no effect on either CDK4 or cyclin D1 mRNA levels (graphs B and C in FIG. 5D, respectively). For CDK2 the differences (SR13904-treated vs. vehicle control-treated) were significant (*p=0.01-0.05, **p=0.00-0.01). Computational analysis of the proximal human CDK2 promoter identified a number of putative consensus PPREs. None were found within the proximal promoter regions of the CDK4 and cyclin D1 genes. SR13904 may directly affect the transcriptional activity of CDK2 while exerting indirect effects on the expression of the CDK4 and cyclin D1 proteins, perhaps through control over PPARδ-dependent signaling pathways. Together, these findings demonstrated the effectiveness of SR13904 in inhibiting progression through the cell cycle and confirmed the importance of PPARδ for this process.

### Example 6

### Apoptosis of Cancer Cells Treated with SR 13904

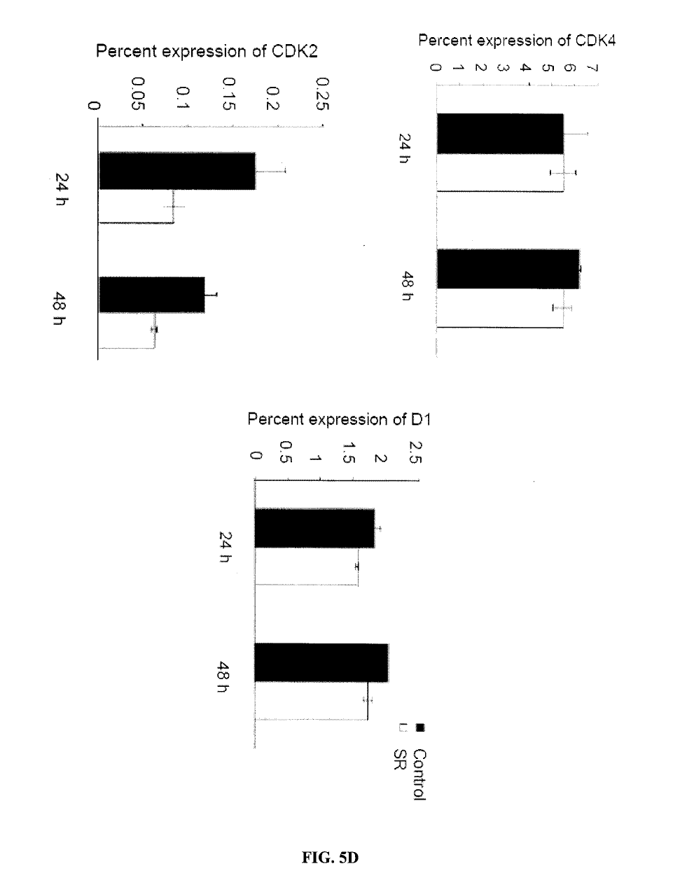

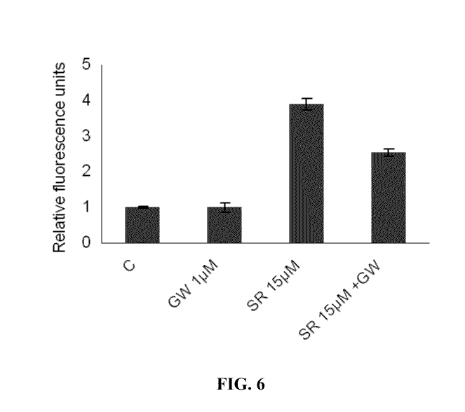

Referring now to FIG. 6, SR13904 treatment of cancer cells results in increased apoptosis. A549 cells (2×104 cells/well) were incubated in 0.5% media (96 well plates) SR13904 for 40 h. The relative degrees of apoptosis were determined using a Cell Death Detection ELISA (Roche). SR represents SR13904. ***p=<0.001 SR vs. SR+GW501.

SR13904 treatment results in increased apoptosis. Down-regulation of PPARδ, either by genetic knockout or by the treatment with 13-S—HODE (15-lipoxygenase-1 product), induces apoptosis in colorectal cancer cells. The effects of SR13904 (15 μM) GW501 (1 μM) on survival of A549 cells (0.5% serum) was determined. Relative degrees of apoptosis were measured using a DNA fragmentation assay. The data shows that SR13904 treatment exerted a significant apoptotic effect on these lung carcinoma cells and that co-treatment with GW501 markedly alleviated this effect.

### Example 7

### SR 13904 Reverses the VLDL-Dependent Increases in MMP-9 Activity of PC-3 Cells

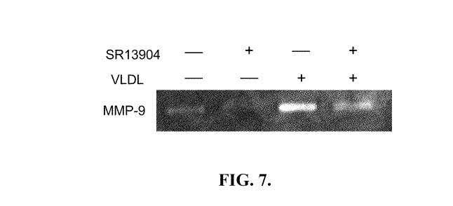

Zymogel studies were performed to determine whether PPARδ could regulate MMP-9 expression/activity in prostate cancer cells. Over-expression of MMP-9 is directly linked to metastatic prostate cancer, at least in part through mitogen/extracellular-signal-regulated kinase kinase 5/extracellular signal-regulated kinase-5 (MEK5/ERK5). An increase in MMP-9 activity in VLDL-treated PC-3 cells was observed (see FIG. 7), and this activation was attenuated by co-incubation with SR 13904.

Referring to FIG. 7, PC-3 cultures were treated with ligands (2 μg/ml VLDL+2 μg/ml LPL and/or 10 μM SR13904) for 16 h prior to harvesting of cells (for cell counting) and collection and concentration of conditioned media. Loading was based on equal cell numbers, corresponding closely to equal loading volumes.

### Example 8

### SR13904 Reverses the VLDL-Dependent Increases in Phosphorylated Akt1 in Growth Factor-Deprived PC3 Cultures

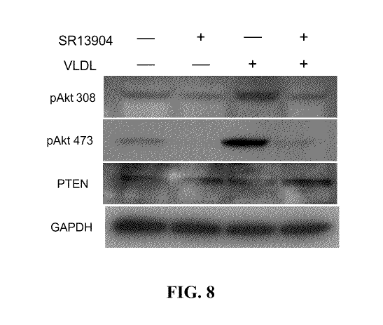

The ability of SR 13904 to regulate the cell survival Akt1 signaling pathway was assessed. PC-3 cultures were exposed to apoptosis-inducing growth factor deprivation and harvested for Western analysis of phosphorylated Akt1 and total PTEN protein. PTEN is known to inhibit the Akt1 pathway at the level of PI3-kinase. Cells exposed to growth factor withdrawal and VLDL displayed relatively high levels of phosphorylation of both serine 473 and tyrosine 308, on the Akt1 protein, as compared with vehicle control (see FIG. 8). Co-treatment with SR 13904 completely abolished the induced phosphorylation of both serine 473 and tyrosine 308. The PTEN blot shown in FIG. 6 shows little or no effect of PPARδ modulation on the expression of this tumor suppressor. Subsequent studies confirmed this conclusion. Regardless, the pAkt data suggest that inhibition of the PPARδ signaling pathway will sensitize prostate tumors to pro-apoptotic stresses or treatments.

Referring to FIG. 8, PC-3 cells were exposed to 48 h of growth factor withdrawal with and without VLDL SR 13904 (2 μg/ml VLDL+2 μg/ml LPL and/or 10 μM SR13904). Standard western analysis for phospho-serine 473, phospho-tyrosine 308, and PTEN were performed. GAPDH was used as a control.

### Example 9

### Binding Assay Data

The LANTHA SCREEN™ TR-FRET PPARδ Competitive Binding Assay was conducted for a variety of compounds according to the instructions provided by the manufacturer (Invitrogen). In addition, proliferation assays were conducted. The data are presented in Table 2. In Table 2, “Prolif IC50” is the concentration that inhibits cell viability (or growth) by 50% relative to a vehicle control, and “TR-F binding IC50 (μM)” is the concentration that competes binding by a labeled pan-agonist to the ligand binding domain of PPAR delta, by 50%. The binding data, in Table 2, demonstrated that 6 of 20 of the compounds bound PPARδ with IC50s in the competition assay between 0.85 and 13.7 μM. No direct correlation could be made between PPARδ binding and inhibition of cell proliferation in this set, since certain compounds with binding IC50>12 μM were active (IC50<10 μM) in the cell proliferation assay.

TABLE 2
Assays with various compounds.

| | | | Prolif | TR-F | |
|---|---|---|---|---|---|
| | | | IC50 | binding | repeat |
| COMPOUND | STRUCTURE | MW | (μM) | IC50 (μM) | TR-F |
| SR13904 | 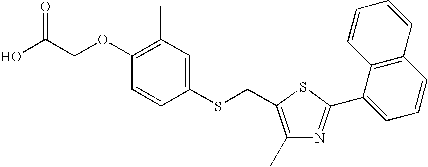 | 459.35 | 12 | 2.4 | 2.1 |
| SRI-010248 | 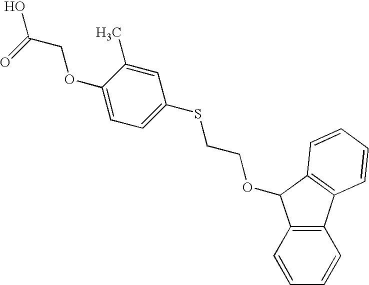 | 406.505 | 9 | >30 | |
| SRI-010249 | 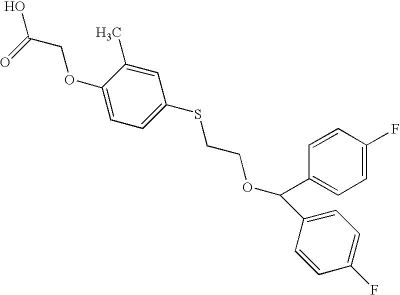 | 444.501 | 5.4 | 0.85 | 1.2 |
| SRI-010250 | 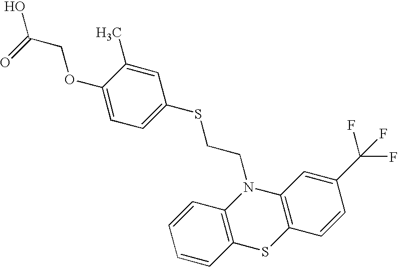 | 491.555 | 7 | 3.8 | |
| SRI-010251 | 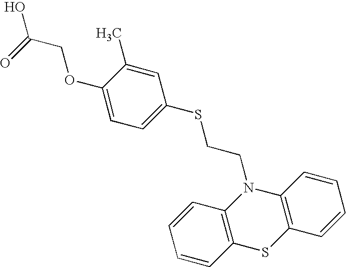 | 423.557 | 7.6 | 13.7 | |
| SRI-010252 | 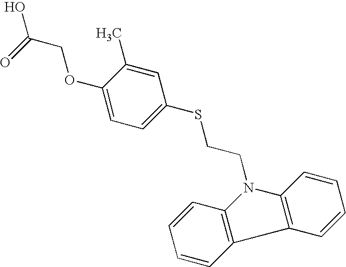 | 391.493 | 9 | >30 | |
| SRI-010253 | 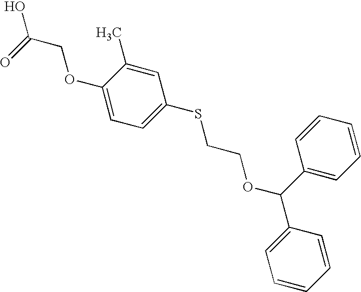 | 408.521 | 14 | 3 | |
| SRI-010254 | 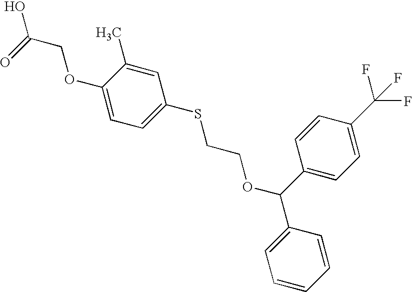 | 476.519 | 7.4 | 1.3 | |
| SRI-010255 | 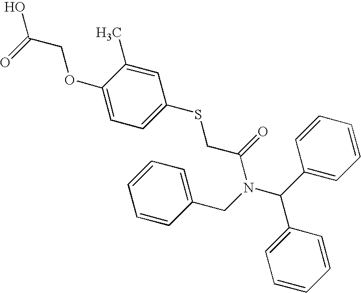 | 511.645 | 5 | *>12 | |
| SRI-010256 | 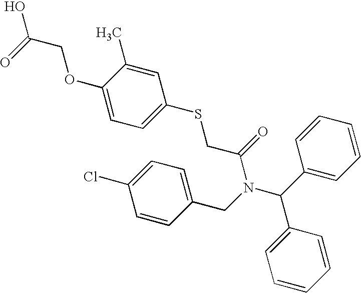 | 546.09 | | *>18 | |
| SRI-010257 | 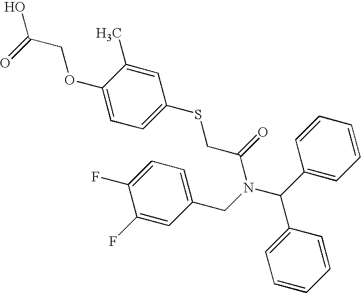 | 547.626 | 9.1 | *>23 | |
| SRI-010258 | 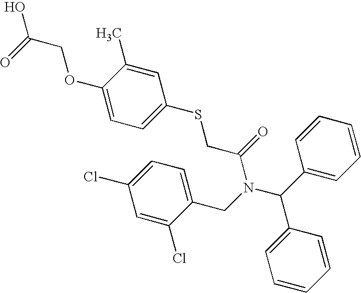 | 580.535 | 4.8 | *>14 | |
| SRI-010259 | 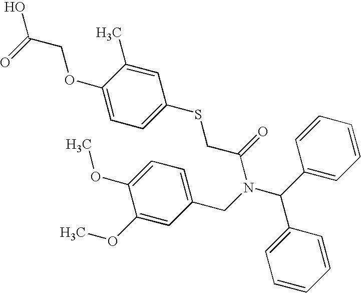 | 571.698 | >40 | *>30 | |
| SRI-010260 | 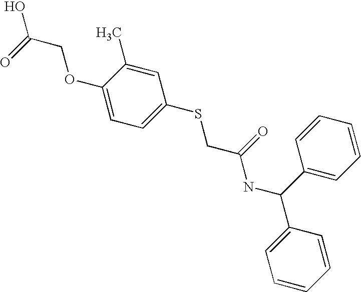 | 421.519 | >40 | not soluble in DMSO | |
| SRI-010261 | 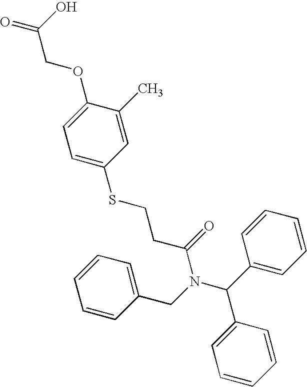 | 525.672 | 8.5 | *>20 | |
| SRI-010262 | 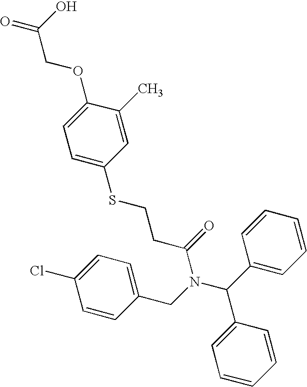 | 560.117 | 5.6 | *>18 | |
| SRI-010263 | 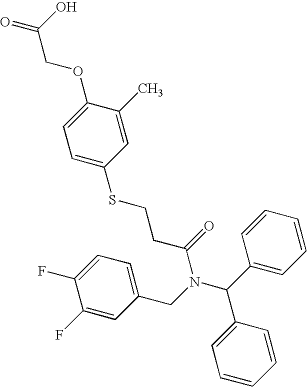 | 561.653 | 9.1 | *>25 | |
| SRI-010264 | 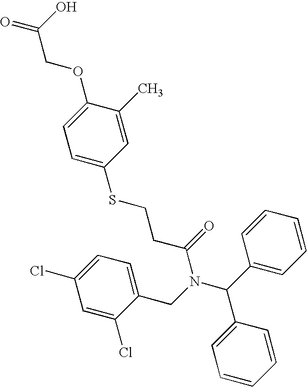 | 594.562 | 5.9 | *>25 | |
| SRI-010265 | 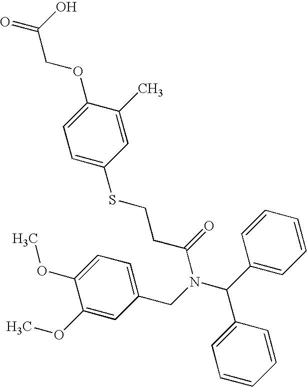 | 585.725 | >40 | *>30 | |
| SRI-010266 | 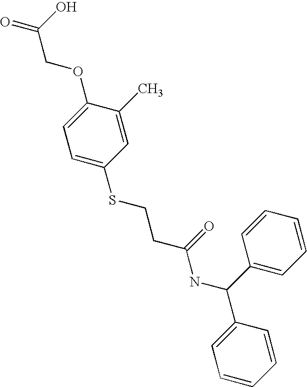 | 435.546 | >40 | *>30 | |

*determined by approximation from a semi log graph

What is claimed is:

1. A method for modulating a PPAR-δ receptor, the method comprising administering a compound having the structure of formula (I): 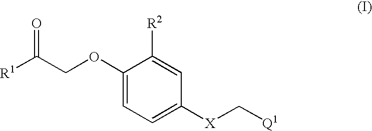
wherein:
R¹ is selected from —OR³ and N(R⁴)(R⁵);
R² is hydrocarbyl;
R³ is selected from H and hydrocarbyl;
R⁴ and R⁵ are independently selected from H and hydrocarbyl;
X is selected from —S—, —O—, and —NR⁸—;
R⁸ is selected from H and hydrocarbyl;
Q¹ is —(CH₂)ₙ-Q²-B
n is an integer from 0 to 3
Q² is selected from a bond, —O—, —C(═O)—NR⁷—, and 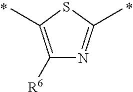
R⁶ is hydrocarbyl;
R⁷ is selected from H, alkyl, aryl, alkaryl, and aralkyl, any of which may be unsubstituted or substituted; and
B is a bulk-providing group.

2. The method of claim 1, wherein the compound has the structure of formula (Ia) 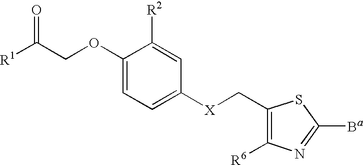
wherein B^{a} is a bulk-providing group that is sterically larger than a 4-(trifluoromethyl)phenyl group.

3. The method of claim 2, wherein B^{a} has the structure 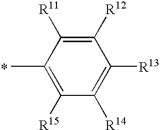
wherein R¹¹-R¹⁵ are H or non-hydrogen substituents, provided that at least two of R¹¹-R¹⁵ are linked to form a cycle.

4. The method of claim 3, wherein R¹ is hydroxyl, R² is methyl, ethyl, or propyl, X is sulfur or oxygen, and R⁶ is methyl.

5. The method of claim 4, wherein B^{a} is selected from naphthyl, substituted naphthyl, heteroatom-containing naphthyl, and substituted heteroatom-containing naphthyl.

6. The method of claim 5, wherein B^{a} is selected from 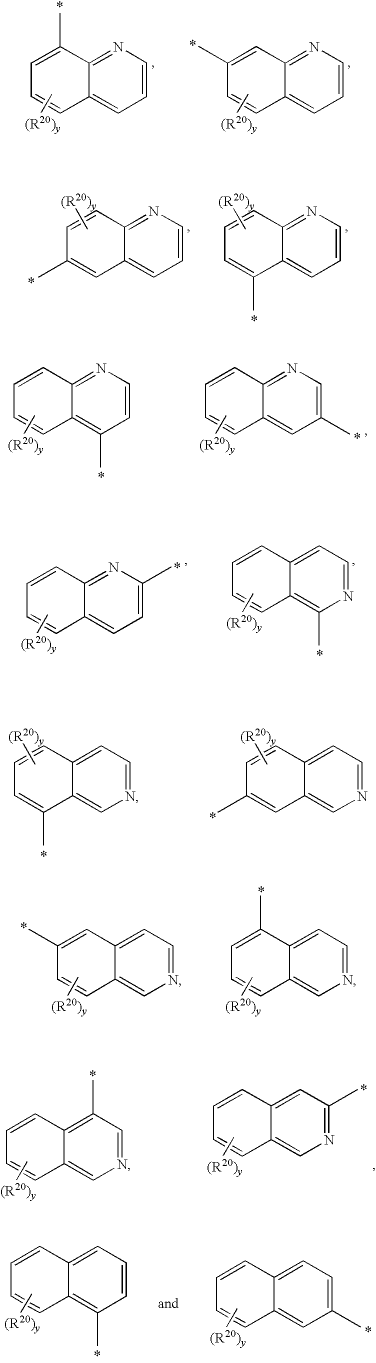
wherein the star represents the attachment point to the remainder of the compound, y is an integer selected from 0, 1, 2, and 3, and each R²⁰ is a non-hydrogen substituent independently selected from alkyl, alkoxy, aryl, and aryloxy, any of which may be halogenated.

7. The method of claim 1, wherein the compound has the structure of formula (Ib) 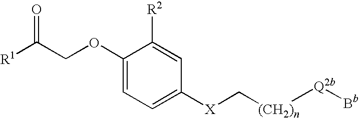
wherein Q^{2b} is selected from a bond, —O—, and —C(═O)—NR⁷—, and B^{b} is a bulk-providing group that is sterically larger than a 4-(trifluoromethyl)phenyl group.

8. The method of claim 7, wherein B^{b} is selected from 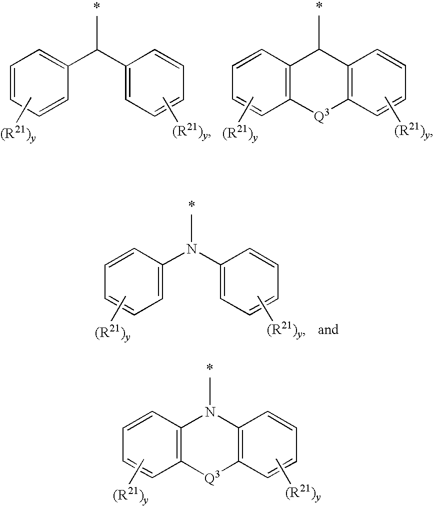
wherein:
the star represents the attachment point to the remainder of the compound;
Q³ is selected from a bond, —S—, and —CR¹⁰R¹¹—;
R¹⁰ and R¹¹ are independently selected from H, lower alkyl, and halo;
each y is an integer independently selected from 0, 1, 2, and 3; and
each R²¹ is halo or a non-hydrogen substituent independently selected from alkyl, alkoxy, aryl, aryloxy, and heteroaryl, any of which may include one or more halo substituents.

9. The method of claim 7, wherein R⁷ is —CH₂—Ar, wherein Ar is a phenyl group that is unsubstituted or substituted with one or more substituents selected from halo, alkyl, and alkoxy.

10. The method of claim 1, wherein the compound is an antagonist or an agonist of PPAR-δ.

11. The method of claim 10, wherein the compound is administered in the form of a pharmaceutically acceptable composition

12. The method of claim 11, wherein the composition further comprises a carrier.

13. The method of claim 1, wherein the method is suitable for modifying a biological process regulated by PPAR-δ.

14. A compound having the structure of formula (I): 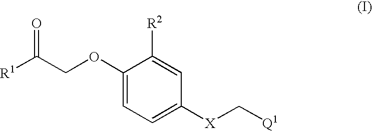
wherein:
R¹ is selected from —OR³ and N(R⁴)(R⁵);
R² is hydrocarbyl;
R³ is selected from H and hydrocarbyl;
R⁴ and R⁵ are independently selected from H and hydrocarbyl;
X is selected from —S—, —O—, and —NR⁸—;
R⁸ is selected from H and hydrocarbyl;
Q¹ is —(CH₂)ₙ-Q²-B
n is an integer from 0 to 3
Q² is selected from a bond, —O—, —C(═O)—NR⁷—, and 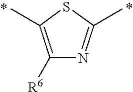
R⁶ is hydrocarbyl;
R⁷ is selected from H, alkyl, aryl, alkaryl, and aralkyl, any of which may be unsubstituted or substituted; and
B is a bulk-providing group that is sterically larger than a 4-(trifluoromethyl)phenyl group.

15. The compound of claim 14, wherein Q² is 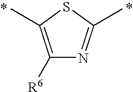
and wherein B has the structure 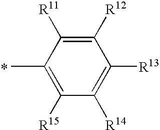
wherein R¹¹-R¹⁵ are H or non-hydrogen substituents, provided that at least two of R¹¹-R¹⁵ are linked to form a cycle.

16. The compound of claim 14, wherein Q² is selected from a bond, —O—, and —C(═O)—NR⁷—, and B is selected from 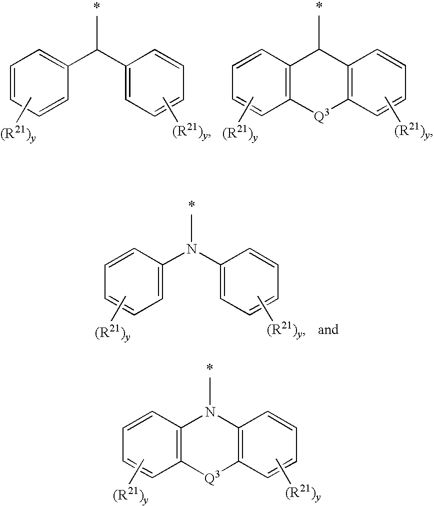
wherein:
the star represents the attachment point to the remainder of the compound;
Q³ is selected from a bond, —S—, and —CR¹⁰R¹¹—;
R¹⁰ and R¹¹ are independently selected from H, lower alkyl, and halo;
each y is an integer independently selected from 0, 1, 2, and 3; and
each R²¹ is halo or a non-hydrogen substituent independently selected from alkyl, alkoxy, aryl, aryloxy, and heteroaryl, any of which may include one or more halo substituents.

17. The compound of claim 14, wherein the compound is an antagonist or an agonist of PPAR-δ.

18. A method for modulating a PPAR-δ receptor, the method comprising administering a compound comprising a trisubstituted thiazole group, wherein the substituent at the 2-position of the thiazole group is a bulk-providing group that is sterically larger than a 4-(trifluoromethyl)phenyl group.

19. The method of claim 18, wherein the bulk-providing group has the structure 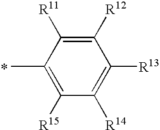
wherein R¹¹-R¹⁵ are H or non-hydrogen substituents, provided that at least two of R¹¹-R¹⁵ are linked to form a cycle.

20. The method of claim 18, wherein the bulk-providing group is selected from 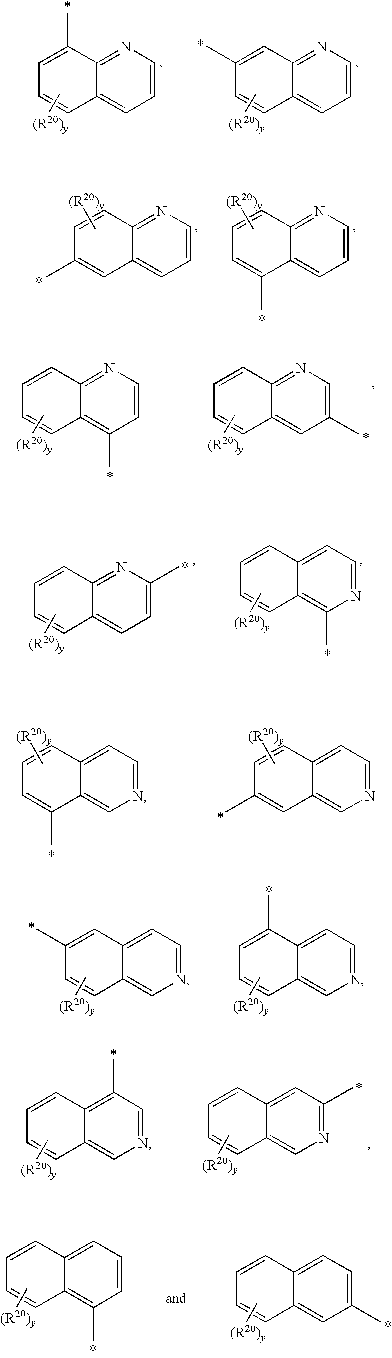
wherein the star represents the attachment point to the remainder of the compound, y is an integer selected from 0, 1, 2, and 3, and each R²⁰ is a non-hydrogen substituent independently selected from alkyl, alkoxy, aryl, and aryloxy, any of which may be halogenated.

21. The method of claim 18, wherein the bulk-providing group is a cyclic group comprising at least two fused rings.
